(19) United States
(12) Patent Application Publication
(10) Pub. No.: US 2026/0232779 A1
(43) Pub. Date: Aug. 13, 2026

(54) FUSION CYTOKINE COMPOSITIONS AND METHODS OF USE THEREOF

(71) Applicant: Wisconsin Alumni Research Foundation, Madison, WI (US)

(72) Inventors: Mahua Dey, Madison, WI (US); Jacques Galipeau, Madison, WI (US)

(73) Assignee: Wisconsin Alumni Research Foundation, Madison, WI (US)

(21) Appl. No.: 19/153,779

(22) PCT Filed: Feb. 6, 2024
(86) PCT No.: PCT/US2024/014638

§ 371 Date: Aug. 5, 2025

Related U.S. Application Data
(60) Provisional application No. 63/483,867, filed on Feb. 8, 2023.

(51) Int. Cl. A61K 39/00; A61P 37/04

(52) U.S. Cl. CPC A61K 39/001139; A61K 39/00114; A61P 37/04; A61K 2039/5152

(57) ABSTRACT

Described herein is an immunogenic composition including a tumor cell expressing a fusokine including GM-CSF linked to IL-7 by a peptide linker. Also described are pharmaceutical compositions and methods of treating patients with glioblastoma with the tumor cell expressing a fusokine including GM-CSF linked to IL-7 by a peptide linker.

### CROSS-REFERENCE TO RELATED APPLICATIONS

This application claims priority to U.S. Provisional Application 63/483,867 filed on Feb. 8, 2023, which is incorporated herein by reference in its entirety.

### FIELD OF THE DISCLOSURE

The present disclosure is related to tumor cells expressing fusion GM-CSF and IL-7 cytokines and methods of treatment of glioblastoma with the tumor cells.

### BACKGROUND

Glioblastoma (GBM), a 100% lethal primary brain cancer is predominately a disease of older adults with median age of diagnosis of 64 years. GBM profoundly influences the host immune system by inducing local and systemic immune cell dysfunction, thus there has been longstanding interest in therapeutic manipulation of GBM's influence on the immune system using immunotherapy. Recent studies point to a broad spectrum of T-cell dysfunction within the tumor microenvironment that renders the durable therapeutic benefit of immunotherapy ineffective. Effector arm insufficiency, characterized by CD8+ T cell dysfunction, tolerance, anergy, exhaustion, and senescence, is a hallmark of inadequate immune response against GBM.

Fusokines, which are engineered through the fusion of two separate and unrelated cytokines, are not bound to physiological regulation and can pharmacologically impel the clustering of unrelated but activated cytokine receptors together. This can result in transducing unique and supraphysiological signals that ultimately confer novel biological effects. GIFT (granulocyte-macrophage colony-stimulating factor and interleukin fusion transgenes) fusokines can modulate immune response, particularly in cancer and autoimmune conditions. Fusokines have been used to enhance the immune system.

What is needed are novel therapeutics for the treatment of GBM, for example.

### BRIEF SUMMARY

In an aspect, an immunogenic composition comprises a tumor cell expressing a fusokine comprising GM-CSF linked to IL-7 by a peptide linker.

Also described are pharmaceutical compositions comprising the immunogenic compositions and methods of treating a patient in need of treatment for a glioma such as a glioblastoma comprising administering the pharmaceutical compositions to the patient.

### BRIEF DESCRIPTION OF THE DRAWINGS

FIGS. 1A-G show GIFT-7 tumor vaccine increases overall survival in aged mice with GBM. 1A) Gating and bar graph quantification for percent of live/CD3+, live/CD3+/CD4+, and live/CD3+/CD8+ cell isolated from PBMC compartment of both VC and GIFT-7 vaccinated mice two- and four-weeks post flank vaccination implantation n=3. 1B) Gating and quantification of the percent of live/CD3+/CD4+/CD44+/CD62L (low) and live/CD3+/CD8+/CD44+/CD62L (low) within mouse PBMC compartment isolated 4 weeks post VC or GIFT-7 vaccination n=3. 2C) Quantity of cytokine detected in pg/μl of conditioned media from the spleen and PBMC compartment of VC and GIFT-7 mice 1 week post intracranial tumor implantation n=3. 1D) Experimental schematic depicting the process of both standard (intracranial only) and vaccination (VC or GIFT-7 vaccination followed by intracranial injection) in-vivo survival experiments. 1E) Kaplan Meyer survival plot of GIFT-7 peripheral vaccination using GL261 tumor cells in aged mice across no immunization (yellow), VC immunization (blue), and GIFT-7 immunization (red). 1F) Kaplan Meyer survival plot of GIFT-7 peripheral vaccination using CT2A tumor cells in aged mice across no immunization (yellow), VC immunization (blue), and GIFT-7 immunization (red). 1G) Immunohistochemistry demonstrating tumor sizes 1 week and 3 weeks post intracranial implantation of GL261 cells in aged mice at both 2× and 10× microscopic resolution. Data are presented as mean±/−SD. Dots in bar graphs (1A/1B) depict individual mice, (1C) depict average cytokine reading from a biological triplicate and technical duplicate. Statistical significance was determined by corrected ANOVA (A/B/E/F) * p<0.05, *** p<0.001, **** p<0.0001.

FIGS. 2A-D show GIFT-7 secretion reduces the size of mouse flank tumor and is ineffective as a peripheral vaccination in young mice. 2A) Bar graph quantification demonstrating secretion of both IL7 and GMCSF in GL261 cells transfected with GIFT-7 plasmid compared to VC plasmid. 2B) Kaplan Meyer curve depicting survival for VC and G7 transfected GL261 cells implanted directly into the intracranial compartment of aged mice. 2C) Gross quantification of flank tumors derived from injected GIFT-7 or VC cells in aged mice. 2D) Kaplan Meyer curve depicting survival of young mice peripherally vaccinated with GIFT-7 or VC cells or receiving no peripheral vaccination. Dots represent technical replicates (2A) or an average of 3 mice with SD depicted as error bars (2C). Statistical significance was determined by corrected ANOVA (2A, C). * p<0.05, **** p<0.0001.

FIGS. 3A-I show GIFT-7 tumor vaccine increases circulating and intratumoral T-cells. 3A) Gating and quantification for percent of live/CD3+, live/CD3+/CD4+, and live/CD3+/CD8+ T-cells isolated from the brains of VC and GIFT-7Tvax mice n=9. 3B) Gating and quantification for percent of live/CD3+, live/CD3+/CD4+, and live/CD3+/CD8+ T-cells isolated from the blood of VC and GIFT-7Tvax mice n=9. 3C) Gating and quantification for percent of live/CD3+, live/CD3+/CD4+, and live/CD3+/CD8+T-cells isolated from the thymus of VC and GIFT-7Tvax mice n=9. 3D) Immunofluorescence imaging of sectioned brains of VC and GIFT-7TVax mice stained with Dapi (blue) anti-CD3 (red) and anti-MHCII (green) at 1 week post intracranial injection. 3E) Gating and quantification of percent of live/CD3+/CD4+/PD1+/Lag3+ cells in VC and GIFT-7TVax mice n=6. 3F) Gating and quantification of percent of live/CD3+/CD8+/GranzymeB+ cells in VC and GIFT-7TVax mice n=6. 3G) Gaiting and quantification of percent of live/CD3+/CD8+/PD1+ cells in VC and GIFT-7TVax mice n=6. 3H) IHC images of dissected thymus from VC and GIFT-7Tvax mice 1 week post intracranial rechallenge. 3I) Gross examination of VC and GIFT-7TVax thymus 1 week post-intracranial tumor rechallenge. Data in bar graphs are presented as mean±/−SD. Dots in bar graphs (3A/B/C/E/F/G) depict individual mice. Statistical significance was determined by corrected ANOVA (3A/B/C/E/F/G) ** p<0.01, *** p<0.001, **** p<0.0001.

FIGS. 4A-C show GIFT-7TVax mice demonstrate rapid mobilization of T-cells to the intracranial compartment upon intracranial tumor implantation. 4A) Gating diagram for follow cytometric isolation of T-cell populations. 4B) Bar graph quantification of CD4+ T-cells at 1 and 3 weeks post intracranial tumor implantation across GIFT-7TVax or VC groups. 4C) Bar graph quantification of the CD8+ T-cells at week 1 and 3 post intracranial tumor injection across GIFT-7TVax or VC groups. Data are presented as mean+/−SD with dots representing individual mice. Statistical significance was determined by corrected ANOVA. * p<0.05, **** p<0.0001.

FIGS. 5A-G show GIFT-7TVax results in increased systemic IL1B and formation of hyperactive dendritic cells. 5A) Schematic representation of cytokine stimulation, QPCR, and immunofluorescence experiments. 5B) Characterization of the broad effects of cytokines contained in the global cytokine screen and pg/μl of detected cytokines from the pro-inflammatory group. 5C) Immunofluorescent staining of isolated spleen dendritic cells from VC and GIFT-7TVax mice 1 week post intracranial challenge. Cells are stained with Dapi (blue), ASC (green), and phalloidin (red). 5D) QPCR quantification (fold change) of NLRP3, Caspase-1, and IL1B, in isolated spleen dendritic cells from VC and GIFT-7Tvax mice 1 week post intracranial challenge n=3. 5E) Schematic of fusokine/cytokine stimulation of BMDC's followed by QPCR and Incucyte movement assay. 5F) QPCR quantification (fold change) of IL1B, Caspase-1, and NLRP3, in BMDC's stimulated with either GIFT-7 (red) or GMCSF+IL7 (blue) n=12. 5G) Line graph, bar chart, and radar plot depicting distance and direction traveled by BMSC's stimulated with GIFT-7 (red) or GMCSF+IL7 (blue) n=30 cells. Data in bar graphs are presented as mean±/−SD. Bars in (5B) depict average cytokine reading from a biological triplicate and technical duplicate. Dots in bar graphs (5D/F/G) depict technical replicates. Statistical significance was determined by corrected ANOVA (5D) and Welsh t-test (5F/G) * p<0.05, ** p<0.01, **** p<0.0001.

FIGS. 6A and B show global cytokine analysis of the thymus and blood of GIFT-7TVax and VC aged mice. 6A) Bar graph quantification of cytokines detected in aged mouse thymus across VC and GIFT-7TVax groups. 6B) Bar graph quantification of cytokines detected in aged mouse blood across VC and GIFT-7TVax groups. Bars depict average cytokine reading from a biological triplicate and technical duplicate.

FIGS. 7A-F show GIFT-7TVax induces RORγt+Th-17 lineage effector memory T-cells and reduces T-cell exhaustion in long-term survivor mice. 7A) Kaplan Meyer survival graph depicting long-term survivor mice (day 125 post intracranial rechallenge) and their sacrifice from the experiment for flow cytometry analysis (day 250). 7B) Gating and quantification of percent of live/CD3+, live/CD3+/CD4+, and live/CD3+/CD8+, T-cells in VC, GIFT-7TVax (1 week post intracranial tumor rechallenge), and long-term survivor mice (125 days post intracranial tumor rechallenge) n=6 (VC/G7TVax) n=3 (long-term survivor). 7C) Gating and quantification of percent of live/CD3+/CD8+/GranzymeB+T-cells in VC, GIFT-7TVax, and long-term survivor mice n=6 (VC/G7TVax) n=3 (long-term survivor). 7D) Gating and quantification of percent of live/CD3+/CD4−/CD8−, live/CD3+/CD4+/CD8+ T-cells in VC, GIFT-7TVax, and long-term survivor mice n=6 (VC/G7TVax) n=3 (long-term survivor). 7E) Gating strategy for live/CD3+/CD4+/Rorgt+T-cells in VC, GIFT-7TVax, and long term-survivor mice isolated from the brain and blood including FMO control. 7F) Quantification of percent positive live/CD3+/CD4+/Rorgt+, live/CD3+/CD4+/Tbet+, and live/CD3+/CD4+/FoxP3+ T-cells in VC, GIFT-7TVax, and long-term survivor mice isolated form the brain and blood n=6 (VC/G7TVax) n=3 (long-term survivor). Data in bar graphs are presented as mean±/−SD. Dots in bar graphs (7B/C/D/F) depict individual mice. Statistical significance was determined by corrected ANOVA (7B/D/F) and Welsh t-test (7C) ** p<0.01, *** p<0.001, **** p<0.0001.

FIG. 8 shows effector memory subtype dominates the T-cell compartment in LTS mice. 8A) Gating diagram for flow cytometric analysis of the memory T-cell compartment in LTS mice. 8B) Bar graph quantification of memory subsets in CD4+ and CD8+ T-cells from the brains of LTS mice. 8C) Bar graph quantification of memory subsets in CD4+ an CD8+ T-cells from the blood of LTS mice. Bars depict individual mice.

FIGS. 9A-F show GIFT-7 vaccination remodels the TCR landscape by increasing overall TCR repertoire clonality during early anti-tumor response. 9A) Bubble plot depicting top 100 TCR clones across VC, GIFT-7TVax, GIFT-7 long-term survivor, young mouse (5 weeks), and aged mouse (52 weeks). Size of bubble represents frequency of clone within population, number within bubble represents number of the clone. 9B) Plot of Simpson clonality across VC, GIFT-7TVax, GIFT-7 long-term survivor, young mouse, and aged mouse. Higher Simpson clonality indicates a less diverse TCR repertoire while lower Simpson clonality indicates a more diverse TCR repertoire. Size of bubble indicates total productive rearrangements within sample. 9C) Ridges plot demonstrating distribution of CDR3 transcript length across VC, GIFT-7TVax, GIFT-7 long-term survivor, young mouse, and aged mouse. Mean CDR3 transcript length is depicted within the ridge bar. 9D) Stacked bar chart visualizing the top 20 productive clonal rearrangements across VC, GIFT-7TVax, GIFT-7 long-term survivor, young mouse, and aged mouse. Each hue change within the plot indicates a unique clone. 9E) Stacked bar chart indicating TCRB gene usage across VC, GIFT-7TVax, GIFT-7 long-term survivor, young mouse, and aged mouse. Usage is represented as a percent of the whole TCRB gene utilization across samples. 9F) Stacked bar chart indicating TCRB gene usage across VC, GIFT-7TVax, GIFT-7 long-term survivor, young mouse, and aged mouse. Bars represent average productive frequency of the TCRB gene across samples. Data are presented as individual clones (9A/D), sample productive rearrangements (9B), and individual TCRB genes (9E/F).

FIGS. 10A-E show low clonality and Th-17 related cytokines are present within GIFT-&TVax mice. 10A) Morisita Index measurement of similarity displayed in a heatmap across all groups. 10B) Raw clonal overlap (purple dots) displayed in a heatmap across all groups. 10 C, D, E) Individual clone tracking between aged and young mice (10C), VC and GIFT-7TVax mice (10D) and LTS and GIFT-7TVax mice (10E). Dots in (10 B, C, D, E) depict individual TCR clones.

FIGS. 11A-G show GIFT-7TVax is therapeutic and increases overall survival in a clinically relevant aged mice model of glioma. 11A) schematic describing in vivo post intracranial implantation GIFT-7TVax as well as G7-Rad. 11B) Kaplan Meyer curve depicting survival for VC immunization post intracranial injection (purple) and GIFT-7Tvax post intracranial injection (orange). 11C) Quantification of cytokine secretion (IL7 and GMCSF) post 50gy radiation treatment in VC and GIFT-7 transfected GL261 tumor cells n=3 (left) and GL261 transfected VC and GIFT-7 cell survival post 10,20,50 and 80 gy of radiation n=6 (right). 11D) Kaplan Meyer survival curve depicting survival for no immunization (yellow), VC immunization (blue), GIFT-7TVax (red), and G7-Rad (green). 11E) Stacked bar chart indicating productive frequency of the top 20 clones across VC, GIFT-7TVax, G7-Rad, and GIFT-7 long-term survivor. Each hue within the plot and group depicts a separate clone. 11F) Ridge plot depicting the mean CDR3 transcript length across VC, GIFT-7TVax, G7-Rad, and GIFT-7 long-term survivor. Mean CDR3 transcript length is displayed within the ridge. 11G) Stacked bar chart indicating the average productive frequency of detected TCRB genes within VC, GIFT-7TVax, G7-Rad, and GIFT-7 long-term survivor. Bars display the average productive frequency of the TCRB gene utilized across the samples. Data are presented as mean±/−SD (11C), individual clones (11E), and individual TCRB genes (11G). Statistical significance was determined by Welsh T-Test (11C left) and corrected ANOVA (C right). **** p<0.0001.

FIGS. 12A-D show low clonality and similarity are present within G7-Rad mice while human PDX lines can be successfully transfected with GIFT-7. 12A) Individual clone overlap (purple dot) depicted on a heatmap between all groups. 12B) Morista Index overlap depicted with a heatmap between all groups. 12C) Single clone tracking between GIFT-7TVax and G7-Rad mice. 12D) Raw nucleotide overlap depicted with Venn diagram across VC, G7-Rad, LTS, and GIFT-7TVax mice. Bars depict mean±/−SD. Statistical significance was determined by corrected ANOVA. **** p<0.0001.

FIGS. 13A and B show the results for GIFT-7TVax tested in two additional transgenic murine glioma cell lines IDHWT_Castro (13A) and SB28 (13B). A significant survival advantage was observed in both models.

FIG. 14 shows results for GIFT-7TVax tested in a cutaneous melanoma model. GIFT-7TVax was generated using a murine melanoma cell line. A significant survival advantage was observed.

FIG. 15 shows a schematic of vaccine production to test a glioblastoma (GBM) patient-specific vaccine response. Surgery day: After surgical resection, GBM cells were isolated from fresh patient tissue. During 3-5 days, neutrosphere cell suspensions were collected. Only well-grown cells were kept for co-culture assay, otherwise this sample was discarded. Day 1: 1 μg Human GIFT-7 and vector control plasmids are transfected to GBM cells (100,000) respectively. Successful transfection can be verified by GFP tag expression. Day 2: Transfected GBM cells are irradiated at 20G. PBMCs are recovered from frozen. Day 3: GBM cells and PBMCs are co-culture at ratio of 1:2 for 3-4 days. All the cells are collected and stained for flow cytometry.

FIGS. 16A-C show the results for the vaccine produced according to FIG. 15. The vaccine resulted in CD8 T-cell proliferation in each patients matched PBMCs.

The above-described and other features will be appreciated and understood by those skilled in the art from the following detailed description, drawings, and appended claims.

### DETAILED DESCRIPTION

GIFT-7 is a fusokine combining the domains of both IL7 and GMCSF with a non-biological linker. Interleukin-7 (IL-7) is critical to the development, proliferation and survival of T-cells in the thymus and has been explored as a supplemental therapeutic option to combat effector arm insufficiency with little clinical success. It has been demonstrated that IL-7 and its interaction with its cognate receptor (IL-7rα) are tightly regulated biologically. This tight physiological regulation necessitates supra-therapeutic doses of the cytokine in order to observe a measurable thymic response, which is not achievable in vivo, limiting clinical effectiveness. Fusokines can aid in this therapeutic challenge because of their ability to bypass typical physiological regulation pathways. For example, GIFT-7 has been shown in aged mice to combat age induced thymic involution resulting in robust production of thymic precursor T-cells. Mice supplemented with systemic GIFT-7 demonstrated thymic cortical hyperplasia and responded with increased CD8+ viral specific T-cells to a cytomegalovirus (CMV) infection.

Described herein is a novel use of fusokines such as GIFT-7 to treat gliomas such as glioblastoma. Biological aging results from the accumulation of a multitude of cellular changes over time resulting in loss of physiological homeostasis and deterioration of various biological systems, including the immune system. Together these changes reshape physiological and immune landscapes and lead to increased vulnerability to diseases. Aging is often the prime risk factor for many severe disorders including cancer, cognitive impairments, movement disorders, and cardiovascular disease. Aging related immune senescence limits the immune system's ability to both recognize and respond to foreign antigens or endogenous cellular distress. Aging is associated with declines in circulating T-cells, antigen presentation, apoptotic cell clearance, as well as thymic atrophy resulting in decreased vaccine efficacy in the elderly. Not all immune cells are uniformly sensitive to aging resulting in a differential impact across several different immune cell compartments. Emerging data from the elderly, suggests that one of the most consequential age-related immune dysfunctions happens within the T-cell compartment. The inventors have unexpectedly found that fusokines such as GIFT-7 can overcome the effects of aging on the T-cell compartment thus making GIFT-7 a novel therapeutic for the treatment of patients in need of treatment for glioblastoma. While compositions described herein are particularly useful in the treatment of older patients, the therapies described herein maybe useful in patients of all ages.

Specifically, described herein are transfections of mouse syngeneic glioma cells (GL261 and CT2A) to produce GIFT-7. The transfected cells both with and without radiation treatment were used as a peripherally administered tumor vaccine. The peripheral GIFT-7 tumor vaccine (GIFT-7TVax) significantly increased overall survival (OS) in GL261 and CT2A aged, but not young, mice with intracranial tumors. Importantly, all the long-term survivor mice rejected contralateral intra-cranial tumor re-challenge. GIFT-7TVax induced thymic regeneration in aged mice resulting in increased T-cell trafficking into the brain tumor microenvironment. The GIFT-7TVax induced anti-tumor immune response was mediated by NLRP3 positive IL-1β producing hyperactivated dendritic cells (DCs). GIFT-7 vaccination limited T-cell exhaustion and global TCR sequencing showed that GIFT-7TVax resulted in focused expansion of tumor related TCR repertoires. GIFT-7Tvax long-term survivors generated durable long-term CD4+ Th-17 memory T-cells. In a clinically relevant model, aged mice vaccinated with irradiated tumor vaccine, derived from irradiated GIFT-7 transfected glioma cells, cleared intracranial tumor implantation 100% of the time. Furthermore, vaccination with GIFT-7TVax post tumor implantation cleared resulted in the clearance of intracranial tumor in >50% of mice. These findings show that in the context of a very aggressive brain cancer, a tumor vaccine incorporating GIFT-7 can combat the effects of aging on the T-cell compartment and boost the efficacy of DCs resulting in a durable long-lasting anti-tumor response.

Described herein are novel immunogenic compositions and methods utilizing fusokines comprising a GM-CSF polypeptide and an IL-7 polypeptide, specifically tumor cells expressing the fusokines. The fusokines are describe, for example, in U.S. Pat. No. 9,375,465.

Interleukin-7 (IL-7) is a γ-chain cytokine that plays a role in T cell development and homeostasis by signaling through its cognate receptor, IL-7R or CD127, and inducing T cell survival and/or proliferation. The fusokine comprises GM-CSF linked to IL-7 by a peptide linker, and this fusion cytokine (fusokine) transgene can be expressed and secreted by mammalian cell lines in a manner that is recognized by both anti-GM-CSF and anti-IL-7 antisera. Exemplary peptide linkers are 5 to 50 amino acids long and may comprise amino acids such as glycine, serine, threonine, asparagine, alanine and proline, such as two or three or more (e.g., up to eight) copies of the sequence Gly-Gly-Gly-Gly-Ser (GGGGS).

A specific fusokine called GIFT7 comprises SEQ ID NO: 1.

(SEQ ID NO: 1)
M W L Q S L L L L G T V A C S I S A P A R S P
S P S T Q P W E H V N A I Q E A R R L L N L S
R D T A A E M N E T V E V I S E M F D L Q E P
T C L Q T R L E L Y K Q G L R G S L T K L K G
P L T M M A S H Y K Q H C P P T P E T S C A T
Q I I T F E S F K E N L K D F L L V I P F D C
W E P V Q E S P V N M F H V S F R Y I F G L P
P L I L V L L P V A S S D C D I E G K D G K Q
Y E S V L M V S I D Q L L D S M K E I G S N C
L N N E F N F F K R H I C D A N K E G M F L F
R A A R K L R Q F L K M N S T G D F D L H L L
K V S E G T T I L L N C T G Q V K G R K P A A
L G E A Q P T K S L E E N K S L K E Q K K L N
D L C F L K R L L Q E I K T C W N K I L M G T
K E H

Also included are fusokines having 80, 81, 82, 83, 84, 85, 86, 87, 88, 89, 90, 91, 92, 93, 94, 95, 96, 97, 98 99 and 99.5% sequence identity to SEQ ID NO: 1 so long as the fusokines are recognized by both anti-GM-CSF and anti-IL-7 antisera.

Percent identities between amino acid or nucleic acid sequences can be determined using standard methods known to those of skill in the art. For instance, for determining the percentage of homology between two amino acid sequences, the sequences are aligned for optimal comparison purposes. The amino acid residues at corresponding amino acid positions are then compared. Gaps can be introduced in one or both amino acid sequence(s) for optimal alignment and non-homologous sequences can be disregarded for comparison purposes. When a position in the first sequence is occupied by the same amino acid residue as the corresponding position in the second sequence, then the sequences are identical at that position. The percent identity between the two sequences is a function of the number of identical positions shared by the sequences, taking into account the number of gaps which need to be introduced for optimal alignment and the length of each gap. The comparison of sequences and determination of percent identity and similarity between two sequences can be accomplished using a mathematical algorithm.

Nucleic acids encoding the fusokines may be used for expression of the fusokines. For example, the GM-CSF and IL-7 encoding sequences can be ligated together in-frame either directly or through a sequence encoding a peptide linker. The GM-CSF-encoding sequence can also be inserted directly into a vector, such as a lentiviral vector or an AAV vector, which contains the IL-7-encoding sequence, or vice versa. Alternatively, PCR amplification of the GM-CSF and IL-7-encoding sequences can be carried out using primers which give rise to complementary overhangs which can subsequently be annealed and re-amplified to generate a fusion gene sequence. SEQ ID NO:2 encodes GIFT-7.

(SEQ ID NO: 2)
ATGTGGCTGCAGAGCCTGCTGCTCTTGGGCACTGTGGCCTGCAGCA
TCTCTGCACCCGCCCGCTCGCCCAGCCCCAGCACGCAGCCCTGGGA
GCATGTGAATGCCATCCAGGAGGCCCGGCGTCTCCTGAACCTGAGT
AGAGACACTGCTGCTGAGATGAATGAAACAGTAGAAGTCATCTCAG
AAATGTTTGACCTCCAGGAGCCGACCTGCCTACAGACCCGCCTGGA
GCTGTACAAGCAGGGCCTGCGGGGCAGCCTCACCAAGCTCAAGGGC
CCCTTGACCATGATGGCCAGCCACTACAAGCAGCACTGCCCTCCAA
CCCCGGAAACTTCCTGTGCAACCCAGATTATCACCTTTGAAAGTTT
CAAAGAGAACCTGAAGGACTTTCTGCTTGTCATCCCCTTTGACTGC
TGGGAGCCAGTCCAGGAGTCACCGGTCAACATGTTCCATGTTTCTT
TTAGGTATATCTTTGGACTTCCTCCCCTGATCCTTGTTCTGTTGCC
AGTAGCATCATCTGATTGTGATATTGAAGGTAAAGATGGCAAACAA
TATGAGAGTGTTCTAATGGTCAGCATCGATCAATTATTGGACAGCA
TGAAAGAAATTGGTAGCAATTGCCTGAATAATGAATTTAACTTTTT
TAAAAGACATATCTGTGATGCTAATAAGGAAGGTATGTTTTTATTC
CGTGCTGCTCGCAAGTTGAGGCAATTTCTTAAAATGAATAGCACTG
GTGATTTTGATCTCCACTTATTAAAAGTTTCAGAAGGCACAACAAT
ACTGTTGAACTGCACTGGCCAGGTTAAAGGAAGAAAACCAGCTGCC
CTGGGTGAAGCCCAACCAACAAAGAGTTTGGAAGAAAATAAATCTT
TAAAGGAACAGAAAAAACTGAATGACTTGTGTTTCCTAAAGAGACT
ATTACAAGAGATAAAAACTTGTTGGAATAAAATTTTGATGGGCACT
AAAGAACACTGA

In another aspect, the fusokine encoding nucleic acid may be in the form of an mRNA such as a synthetic mRNA suitable for expressing the fusokine, e.g., GIFT-7.

To prepare the immunogenic compositions, e.g., vaccines, the vector expressing the fusokine, e.g., GIFT-7, or a synthetic mRNA encoding the fusokine is transfected into a tumor cell using, for example, Lipofectamine™, electroporation, lipid nanoparticles, and other means known in the art for introducing nucleic acids into tumor cells. Tumor cells express multiple tumor-associated antigens (TAAs) and/or tumor specific antigens (TSAs) and have been used as vaccines to stimulate anti-tumor immunity.

In an aspect, the tumor cell is a cultured tumor cell such as a tumor cell line. The term tumor cell line refers to a cell line that originated from a cancerous tumor as described herein, and/or originates from a parental cell line of a tumor originating from a specific source/organ/tissue such as a cancer stem cell line. In certain embodiments, the tumor cell line includes a cell line following any number of cell passages, any variation in growth media or conditions, introduction of a modification that can change the characteristics of the cell line such as. As used herein, the term “cell line” also encompasses genetically homogeneous cell lines, in that the cells that make up the cell line(s) are clonally derived from a single cell such that they are genetically identical. This can be accomplished, for example, by limiting dilution subcloning of a heterogeneous cell line. The term “cell line” also encompasses any genetically heterogeneous cell line, in that the cells that make up the cell line(s) are not expected to be genetically identical and contain multiple subpopulations of cancer cells.

Tumor cell lines can be autologous (from the patient) or allogenic (from a donor).

In another aspect, the tumor cell is an autologous cell from a tumor sample from the patient, that is, a cell from a fresh tumor sample prepared from a surgical resection of a tumor. Unlike a tumor cell line, an autologous cell from a tumor sample from the patient is not passaged prior to transfecting with the fusokine comprising a GM-CSF polypeptide and an IL-7 polypeptide.

In an aspect, the tumor cell is non-proliferating. The tumor cell can be irradiated or subjected to gene editing to prevent proliferation. Exemplary irradiation conditions include ex vivo irradiation at 50 Gy to 100 Gy. Irradiation can take place in controlled lab conditions (with a clinical irradiator such as a linear accelerator or cesium irradiator) prior to delivery to patients. Alternatively, gene editing using CRISPR-Cas9, for example, can be used to inactivate genes responsible for tumor cell proliferation.

Also included herein are pharmaceutical compositions comprising the tumor cells expressing the fusokine and a pharmaceutically acceptable carrier. Pharmaceutically acceptable carriers include, but are not limited to, large, slowly metabolized macromolecules such as proteins, polysaccharides, polylactic acids, polyglycolic acids, polymeric amino acids, amino acid copolymers, inactive virus particles, and the like. Pharmaceutically acceptable salts can also be used in the composition, for example, mineral salts such as hydrochlorides, hydrobromides, phosphates, or sulfates, as well as the salts of organic acids such as acetates, proprionates, malonates, or benzoates. The composition can also contain liquids, such as water, saline, glycerol, and ethanol, as well as substances such as wetting agents, emulsifying agents, or pH buffering agents. Liposomes can also be used as carriers.

A method of treating a patient in need of treatment for a glioma comprises administering to the patient the pharmaceutical composition comprising the tumor cells expressing the fusokine and a pharmaceutically acceptable carrier. In an aspect, the patient is 50, 55, 60 or 64 years of age or older. Unexpectedly, the tumor cells expressing the fusokine described herein are particularly effective in targeting the aged immune system.

In an aspect, the glioma is a glioblastoma. Glioma is a broad term for cancers of the glial cells that surround nerve endings in the brain. Types of gliomas include astrocytomas, ependymomas, oligodendrogliomas and glioblastomas.

In an aspect, the patient is a mammalian patient such as a human patient.

In an aspect, the administering is by peripheral injection, such a subdermal or intramuscular injection.

In an aspect, a patient has been diagnosed with glioblastoma has been treated by the current standard of care, which is surgical resection, followed by radiation and optionally chemotherapy. Chemotherapy for glioblastoma includes temozolomide, carmustine, bevacizumab, lomustine, and combinations thereof. Additional co-therapies include administration of immune checkpoint inhibitors and stereotactic radio surgery. Immune checkpoint inhibitors include ipilimumab, cemiplimab, nivolumab, pembrolizumab, and atezolizumab. Stereotactic radiosurgery (SRS) uses many precisely focused radiation beams to treat tumors.

In an aspect, treatment with the immunogenic compositions described herein reduces intracranial tumor growth, provides an anti-tumor immune response (increase in CD3+ T-cells, CD4+ T-cells, CD8+ T-cells, effector memory T-cells, Il-2, Il-4, IL-1β, Caspase-1, NLRP3), or an provides an increase in overall survival.

The invention is further illustrated by the following non-limiting examples.

### EXAMPLES

### Methods

Animal Housing and Survival Experiments: All animals used in experiments were maintained in accordance with the IRB and IACUC committee rules established at the University of Wisconsin. C57/Black 6 mice used for research were obtained from Charles River laboratories. Ages of the mice used in the experiments are noted specifically within the results section and ranged from 6-87 weeks. All mice were contained in a temperature-controlled animal room maintaining a 12-hour light and dark cycle.

For tumor implantation and survival experiments, cells were removed from culture plastic with trypsin and confirmed to be in single cell suspension. The suspension mixture was then counted using a Biorad cell counter with Trypan blue dye used to exclude dead cells. Once the desired count of cells was obtained a solution containing Phosphate Buffered Saline along with the appropriate number of cells was created. Under sterile conditions in a biosafety hood, mice were anesthetized using ketamine, the scalp was shaved and cleaned, and an incision and burr hole were made to the skull. A maximum of 5 μl of cell suspension was injected into the brains of the mice using a stereotactic frame with coordinates 2 mm behind the bregma suture and 1.5 mm posterior to the midline suture. The skin incision was closed using sterile vicril sutures and mice were given post procedure analgesics (buprenorphine) in order to ease discomfort.

In all animal experiments a straight 50/50 breakdown of males and females was maintained.

For flank vaccination cells were obtained as described above and injected into the right flank of the desired mouse using a sterile insulin syringe with no injection volume exceeding 150 μl. Flank tumors were monitored throughout the life of the mouse and were not allowed to exceed veterinary defined sizes or become ulcerated/necrotic. Flank tumor was measured at indicated times using calipers and recorded and plotted using Graphpad Prism.

Cell Culture and Dendritic Cell Isolation: All cells utilized for experimentation were maintained in a sterile culture within a Thermo Fischer Vios cell incubator at 37° C. with 5% CO₂ supplementation. Mouse tumor lines GL261 and CT2A were maintained with DMEM supplemented with 10% fetal bovine serum and 5% penicillin/streptomycin antibiotic. Dendritic cells were maintained in RPMI supplemented with IL4 (10 ng/ml), GMCSF (10 ng/ml) and Glutamax™ (100×) growth factors as well as beta-mercaptoethanol (1 ng/ml) and penicillin/streptomycin antibiotic (5%). Dendritic cells were isolated from mouse bone marrow progenitor cells as per methods known in the art. Briefly, mouse femurs were dissected, and muscle removed under sterile conditions, a break in the femur was induced on each side and marrow flushed out using blank RPMI through an insulin syringe. Marrow was then broken up via pipetting to create a single cell suspension which was then plated on non-adherent tissue culture dishes utilizing the above media recipe. Dendritic cells were allowed to form for progenitors for 7 days before use in experiments.

Immunohistochemistry and Immunofluorescence: IHC was carried out on 8-micron sections of frozen brains after an overnight soak in a sucrose solution. Sections were mounted to cover-glass and stained with hematoxylin and eosin and then sealed for brightfield imaging on a Keyence microscope. For immunofluorescence on mouse brain sections, the sections were blocked using goat serum and then stained in with surface antibodies for MHC-II and CD3 overnight. After overnight staining the antibodies were washed away and the sections were sealed for visualization on a Keyence fluorescence microscope.

Fusokine Transfection: Lentiviral constructs for fusokine expression were obtained and transfected into both GL261 and CT2A using a lentiviral vector system and Lipofectamine™ transfection reagent. Puromycin selection and flow cytometric sorting was utilized to purify a transfected population. For fusokine transfection in human GBM patient samples a similar method was utilized without the inclusion of the puromycin selection due to observed cell line toxicity. Successful transfection was confirmed by both fluorescence staining (GFP tag contained in lentiviral construct) and ELISA for component cytokines (GM-CSF/IL-7). ELISA kits were purchased from Thermo and utilized according to manufacturer's instruction and readout was visualized on a Molecular Devices fluorescent plate reader.

Inflammasome Formation Assays: For stimulation with fusokine or cytokines, dendritic cell media was removed and blank RMPI containing the necessary cytokine was added. Fusokine stimulation on dendritic cells was carried out as described in the art. Dendritic cells were incubated with fusokine/cytokine for 3 hours and then activated with lipopolysaccharide (1 μg/ml) overnight. Before collection for experiments dendritic cells were also primed with ATP (1 μg/ml) 10 minutes prior to collection. Once collected, activated dendritic cells were lysed and RNA was collected (Quiagen RNeasy®) for QPCR as per standard protocols (Applied Biosystems Instrumentation). Primers were designed using Primer Blast (NCBI) and de-novo synthesized by Thermo-Fischer.

For IF inflammasome visualization cytokine/fusokine stimulation was carried out as above. Cells were then fixed (PFA), blocked (Goat Serum), and stained with anti-ASC (find), Phalloidin (Sigma), and Dapi mounting media (Fischer). Slides were visualized on a Keyence fluorescence microscope.

Dendritic Cell Movement Assay: Dendric cell movement post cytokine/fusokine stimulation was visualized using an Incucyte® (Sartorius) incubator within a standard cell culture incubator. Fusokine/cytokine stimulation was carried out as described above. Stimulated and activated dendritic cells were serially photographed every 2 hours for 20 hours and the resulting image files were stitched together using Fiji. Manual cellular movement tracing was conducted in Fiji for n>30 cells in each condition. Traces from two independent individuals were averaged to create a resulting data file that was plotted in both R and Excel. Radar charts were generated in excel using data from >30 dendritic cell traces compiled in Fiji and normalized to start at a common center point.

Flow Cytometry: Flow cytometry was carried out as described in the art. Briefly, mice were sacrificed, and the relevant organs were dissected out and filtered through 70 micro mesh creating a single cell solution. The lymphocyte population was enriched in brain samples using a percoll density centrifugation gradient. Blood lymphocytes were enriched utilizing an ACK lysis buffer treatment to remove red blood cells. Cellular staining was carried out according to antibody manufacturer's instructions (antibody list included in supplemental materials). Design of flow cytometry staining panels was checked for fluorophore overlap using Thermo's panel design tool. Flow cytometry was carried out an Attune™ flow cytometers (Thermo) with multi-antibody compensation as well as FMO controls. Analysis gates were set based on live lymphocytes (Fixable Yellow Viability Dye Thermo) and then relevant FMO control. Flow analysis was conducted in Flowjo with results plotted using Graphpad Prism.

Global Cytokine Screening: Global cytokine screening was conducted using Isoplexis Spark instrumentation along with Isoplexis Mouse Adaptive Immune assay chip according to the published manufacturer's protocol (https://isoplexis.com/support/sample-preparation/). Briefly, organs were isolated from sacrificed mice in the various treatment groups and filtered through 70-micron mesh to create single cell solutions. Cells were plated in triplicate in 96 well dishes and subject to PMA/Ionomycin stimulation for 24 hours prior to cytokine analysis. Pooled conditioned media from isolated triplicates was loaded onto the Isoplexis mouse adaptive immune chip which was then run on the Isoplexis spark. Cytokine level analysis was checked against background levels using Isoplexis quality control metrics on the Isospark machine. Result measurements form the analysis represent a biological triplicate of conditioned media per organ analyzed as well as an average of a technical duplicate run on the chip itself. All reported effects were marked as significant between groups and above background detection by the Isospark instrument. Results were pulled from the Isospark software and plotted using Graphpad.

T-cell Receptor Sequencing: TCR sequencing was carried out by Adaptive Biotechnologies according to standard company protocols. Experimentally, mice from each group were sacrificed and blood draw was obtained through cardiac puncture. Isolation of genomic DNA was performed in the laboratory using a Quiagen DNeasy® Blood and Tissue Kit and gDNA quality and concentration was confirmed using a NanoDrop™ fluorimeter (Thermo). Isolated gDNA was then sent to Adaptive headquarters where proprietary primer sets were utilized to isolate and amplify the TCRB gene locus within the mouse genome. Sequencing of the resulting cDNA libraries was performed at Adaptive's facility and PCR duplication bias was eliminated using established company quality controls. Both raw and quantified data was transferred back to the lab using Adaptive's data analysis cloud. Raw data was analyzed using MiXCR while quantified data was mined and plotting using R (version 4.1).

Cellular Radiation: Radiation of cells was done in a contained and biologically shielded gamma radiation generating instrument within the Small Animal Radiation and Imaging Facility at UW-Hospitals. Cells were radiated well adhered to their culture dishes and given 24 hours to recover prior to standard tissue culture collection for use in experiments. Doses of radiation for cells lines was calculated by the radiation machine software according to size and shape of culture dishes. The same instrument and radiation dosing protocol were used throughout the entire study to minimize variation in radiation doses received by cells. In vivo injection of radiated cells was carried out as described in the animal use section.

Statistical Analysis: Statistical analysis for the project was conducted using both R (version 4.1) and Graphpad Prism. Biorender (Biorender.com) was used for schematic diagram and graphical abstract creation. Where bar graphs are used individual data points are shown with the bar location representing the mean of the data set and the standard deviation represented by error bars. Calculated p-values are indicated using stars (* p<0.05, ** p<0.01, ***p<0.001, ****p<0.0001). Welsh T-tests were used to compare between two groups (assuming unequal standard variance within the data), while ANOVA with Bonferroni correction was utilized to compare between more than 2 groups. Survival data was obtained using the Kaplan Meyer method with multiple group comparison correction (Bonferroni). Plots of survival data indicate median survival as calculated by Graphpad. Statistics for QPCR analysis relied on quantification by the delta-delta CT method followed by either t-test or multiple comparison corrected ANOVA in Graphpad. For Flow cytometry graphs each data point represents an individual mouse (technical replicates) and each experiment was replicated fully at least once (biological replicates) with n>3 mice (separated evenly by gender). Statistics for TCR sequencing were supplied with the quantified data files transferred from adaptive because of their proprietary bioinformatics method for correction of PCR duplication errors. R and the ggplot package were utilized to visualize measurements provided by adaptive such as productive clonality, Simpson clonality, distributed CDR3 length, clone productive frequency, and TCR beta gene usage. Simpson clonality measurements across multiple samples were compared and corrected using Dunn's post-hoc correction. Standard deviations of transcript length or TCRB gene usage were testing using a two-way F test. Productive clonality and TCRB gene usage were analyzed by comparing the mean productive clonality between top 20 clones, or TCRB gene productive frequency, per sample using 2-way ANOVA with Tukey correction for multiple comparisons.

### Example 1: Gift-7 Tumor Vaccine Increases Overall Survival in Aged Mice with GBM

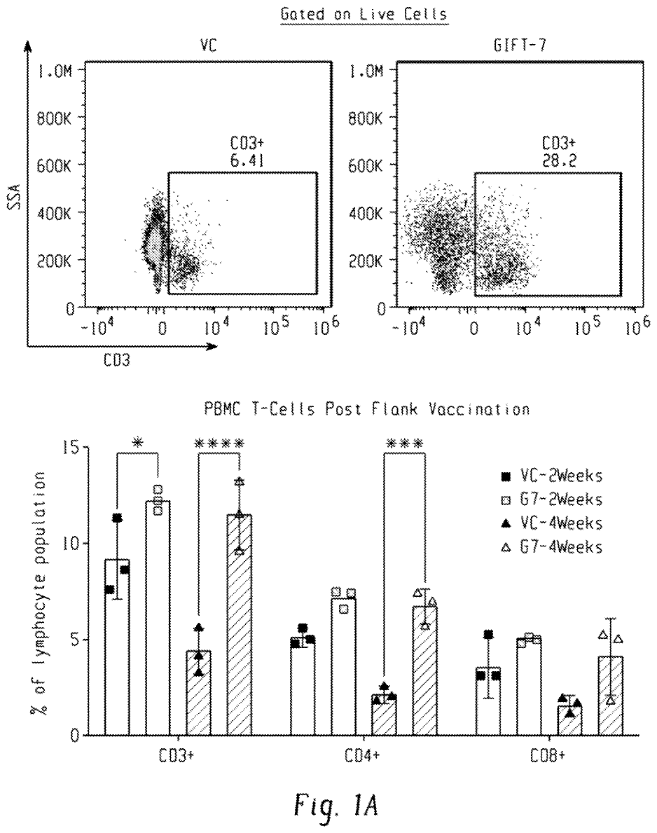

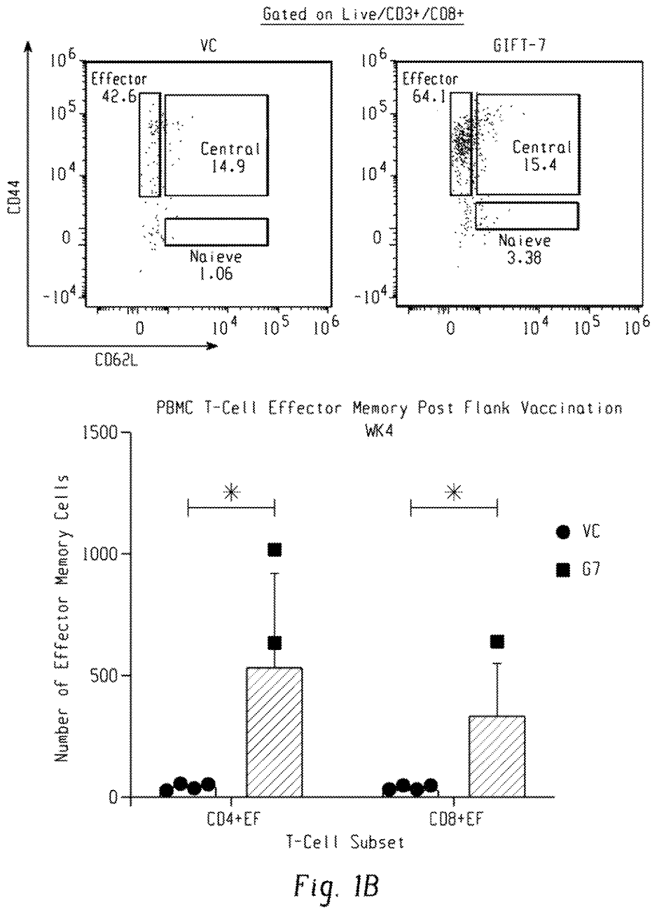

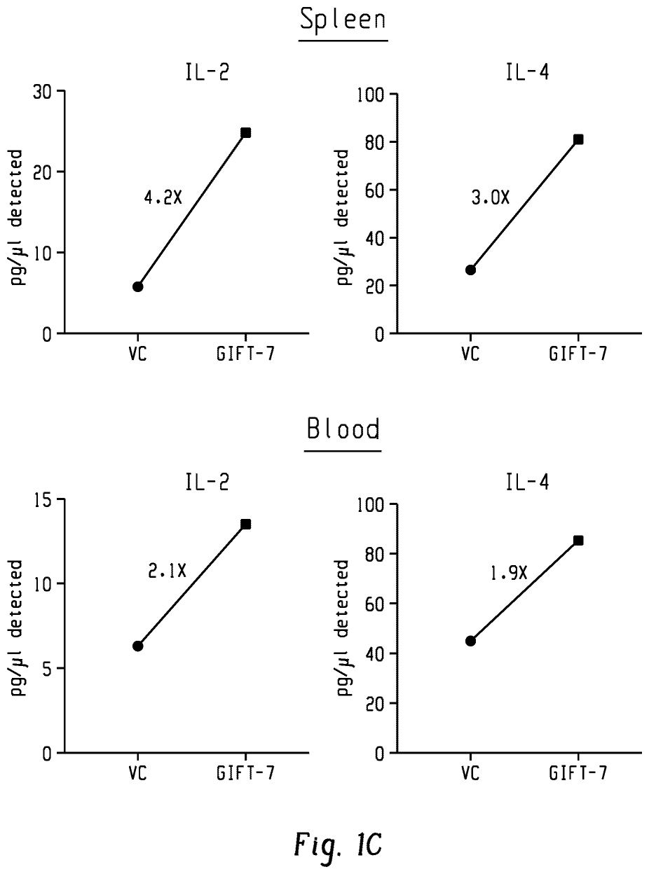

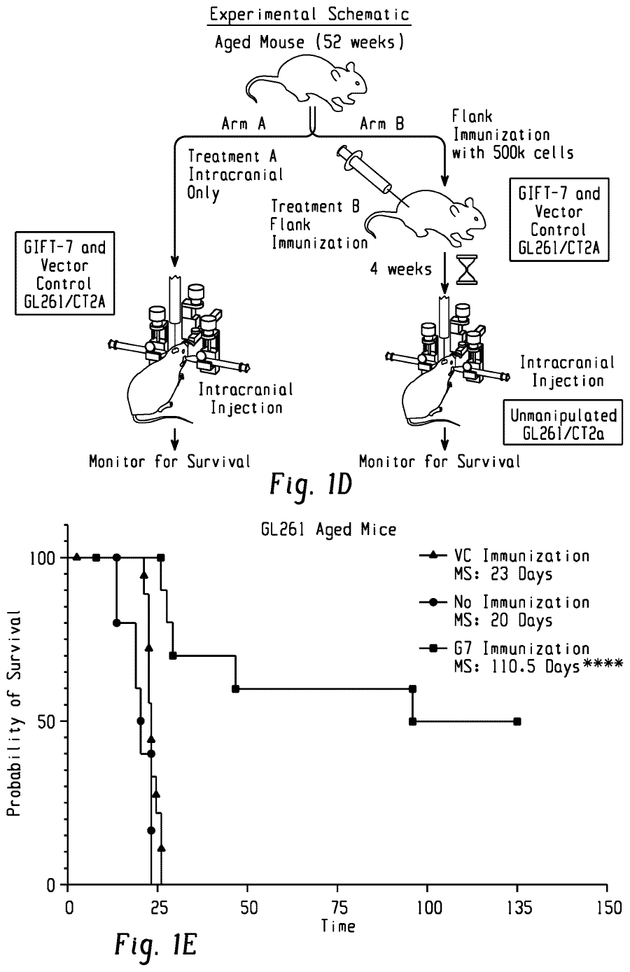

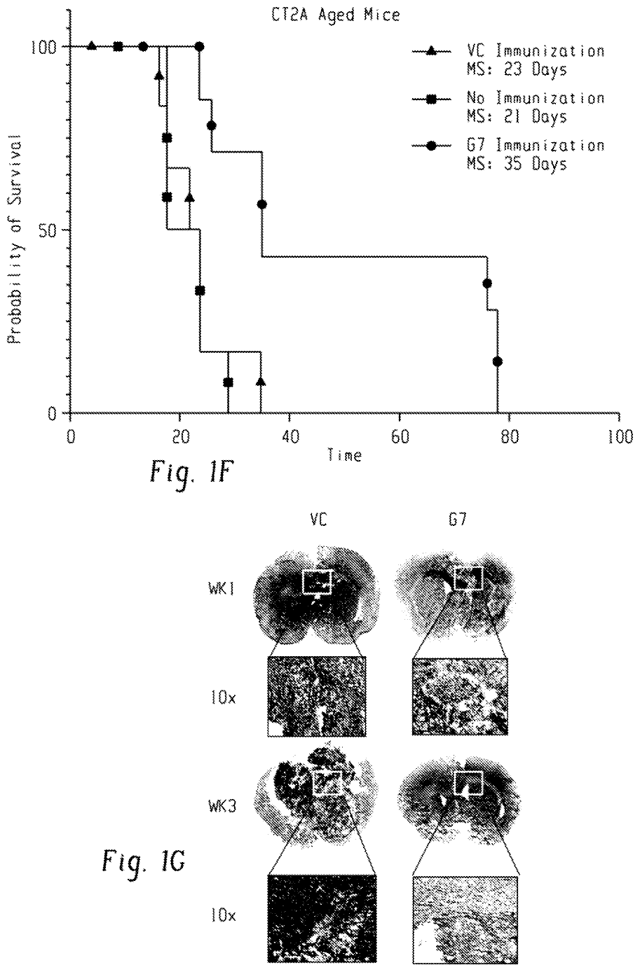

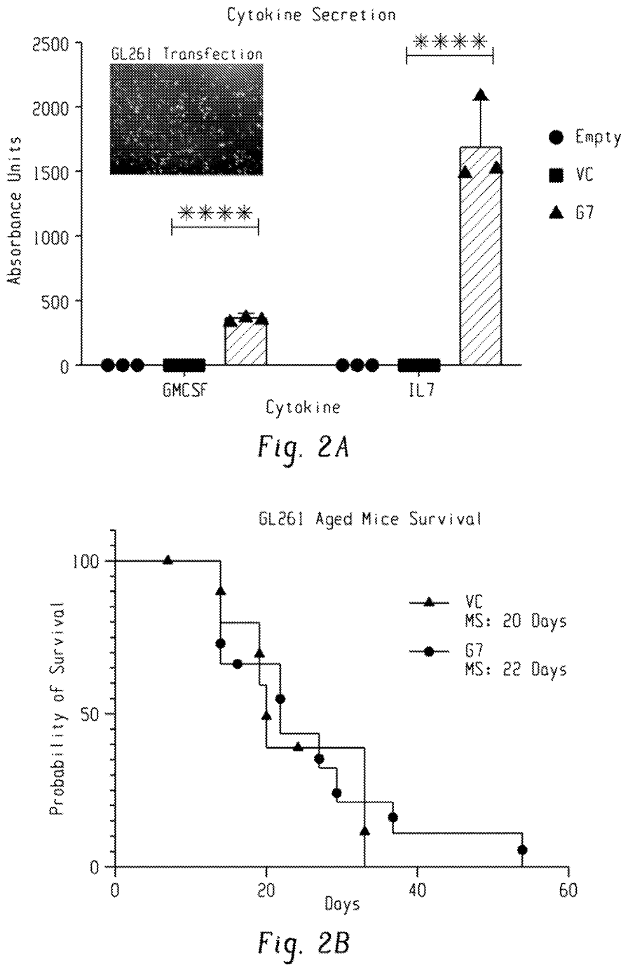

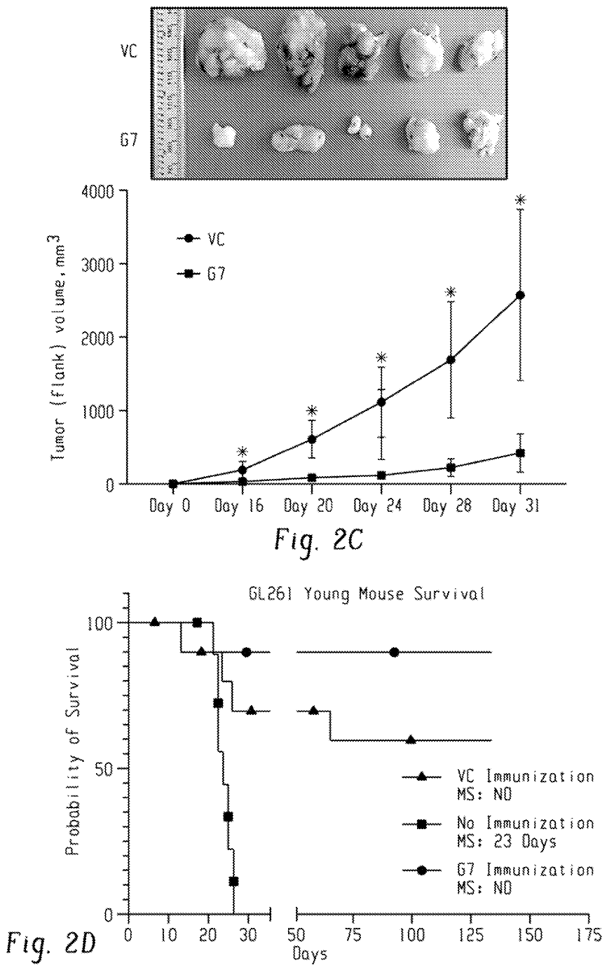

Since GIFT-7 has been shown to reconstitute the aging thymus and increase virus specific effector T-cells, it was first sought to understand the impact of the GIFT-7 fusokine locally on the intracranial tumor growth and anti-tumor immune response in an aged (52 weeks or above) syngeneic mouse model of glioma. To accomplish this, mouse glioblastoma lines GL261 and CT2A were transfected with GIFT-7 plasmid allowing for endogenous production of the fusokine by the GBM cells (GL261GIFT-7 vs GL261VC: GMCSF: 250 absorbance units (AU) vs. 5.69 AU p<0.0001; GL261GIFT-7 vs GL261VC: IL7: 1056.66 AU vs. 0 AU p<0.0001) (FIG. 2A). GL261 cells transfected with GIFT-7 (GL261GIFT-7) and VC (GL261vc) were then injected intracranially into the brains of C57/BL6 mice and were monitored for overall survival (OS). No statistically significant difference in the OS was seen between the two groups (median survival (MS) VC 20 days Vs GIFT-7 22 days) (FIG. 2B). Without being held to theory, it was hypothesized that the locally produced GIFT-7 may have limited systemic presence due to the blood brain barrier. This hypothesis was tested by implanting GL261GIFT-7 and GL261vc in the flank of C57/BL6 mice. Significantly smaller flank tumors were observed in mice with GL261GIFT-7 tumor cells compared to GL261vc (FIG. 2C). In this flank tumor model, analysis of the peripheral blood at 2-weeks and 4-weeks post injection showed significantly higher numbers of overall CD3+ and CD4+ T-cells in the GL261GIFT-7 group compared to GL261 vc (VC vs GIFT-7 2-weeks: CD3+: 9.07% Vs 12.27% p<0.05; 4-weeks: CD3+: 4.4% Vs 11.6% p<0.0001; CD4+: 2.2% Vs 6.8% p<0.001) (FIG. 1A). T-cell phenotype analysis showed a significantly higher number of effector memory T-cells at 4-weeks after flank implantation of GL261GIFT-7 compared to GL261vc (VC vs GIFT-7: Effector Memory CD4+: 43 cells vs 537 cells p<0.05; Effector Memory CD8+: 28 cells vs 328 cells p<0.05) (FIG. 1B). A global cytokine screen conducted at a similar timeframe also concluded that two cytokines involved in the formation and maintenance of immunological memory, IL-2 and IL-4, were increased within both the spleen and blood of mice receiving GL261GIFT-7 (Spleen IL-2 4.2×, Spleen IL-4 3.0×, Blood IL-2 2.1×, Blood IL-4 1.9×) (FIG. 1C). Given the significant difference in flank tumor growth between the treatment groups and the increase in overall T-cells, specifically effector memory T-cells present in the peripheral blood of mice with GL261GIFT-7 flanks, it was studied if the peripheral anti-tumor immune response was reflected in the intracranial compartment post flank tumor implantation. To this end, a peripheral vaccination model was employed where aged mice were vaccinated in the flank using a smaller number of GL261GIFT-7/GL261vc cells and CT2AVC/CT2AGIFT-7 cells. Following vaccination, after 4 weeks and intracranial tumor implantation proceeded using the unmodified respective parental cell lines. To delineate the effect of peripheral immunization with VC tumor cells on the intracranial tumor, a group of mice that received only intracranial tumor without any prior vaccination, and monitored all the groups for OS (FIG. 1D). In both the GL261 and CT2A cell line, groups vaccinated with GIFT-7 modified tumor cells (GIFT-7TVax) demonstrated a significantly increased OS when compared to VC group (MS: 23 days vs 35 days CT2AVC vs GIFT-7Tvax p<0.05; MS: 23 days vs 110.5 days GL261VC 155 vs GIFT-7Tvax p<0.0001) (FIGS. 1E and F). In both the GL261vc and CT2Avc peripheral vaccination groups there was no statistically significant change in OS when compared to the unvaccinated group, indicating no peripheral vaccination effect was present when unmodified cells were given. (MS: 23 days Vs 21 days CT2AVC vs. no vaccination p=NS; MS: 23 days Vs 20 days GL261VC vs no vaccination p=NS) (FIGS. 1E and F). More importantly, in the GL261 model, 50% of mice receiving GIFT-7Tvax became long-term survivors lasting 125 days post intracranial tumor implantation, demonstrating full tumor clearance (FIG. 1E). H&E staining of the brains of mice 1-week and 3-weeks post intracranial tumor implantation from the GL261VC and GIFT-7Tvax group showed smaller tumor volume in the GIFT-7Tvax group at 1-week and complete clearance of the tumor by 3-weeks (FIG. 1G). To delineate the age specific effect of GIFT-7, the tumor vaccination strategy was repeated in 6-8 week old mice, which is the current conventional age of mice used for GBM rodent models. Interestingly, in the younger mouse model, peripheral vaccination with just tumor cells significantly increased OS compared to the unvaccinated group (MS, no vaccination vs vaccination: 23 days vs ND) and GIFT-7Tvax had no additional survival advantage compared to the VC vaccine group (FIG. 2D). Taken together, these findings show that in an aging mouse model of GBM, peripheral tumor vaccine in combination with GIFT-7 can significantly slow the growth of intracranial tumor in a less immunogenic CT2A model and can completely irradicate intracranial tumor in 50% of mice in the more immunogenic GL261 model.

### Example 2: Gift-7 Tumor Vaccine Increases Circulating and Intratumoral T-Cells

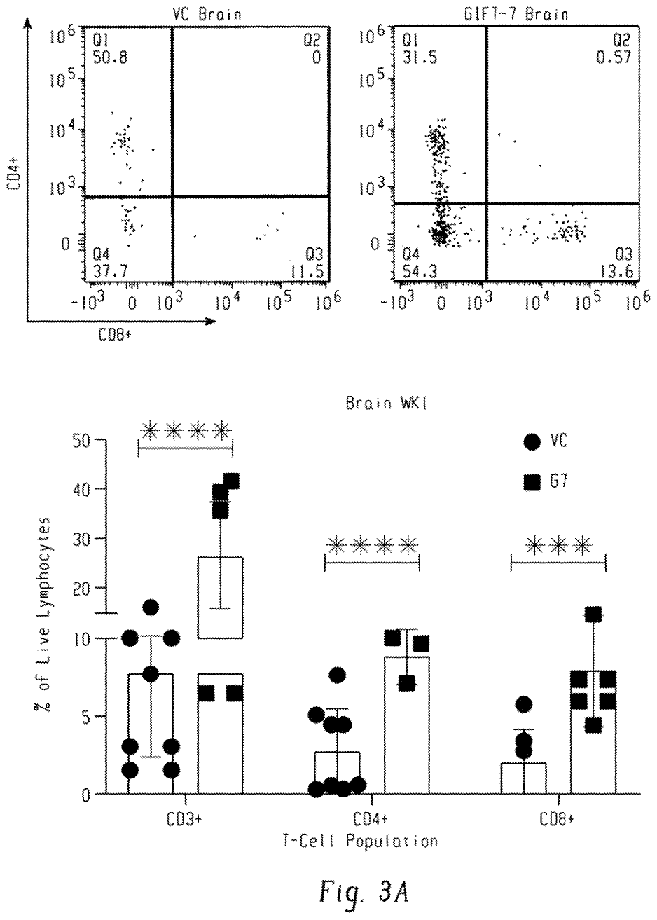

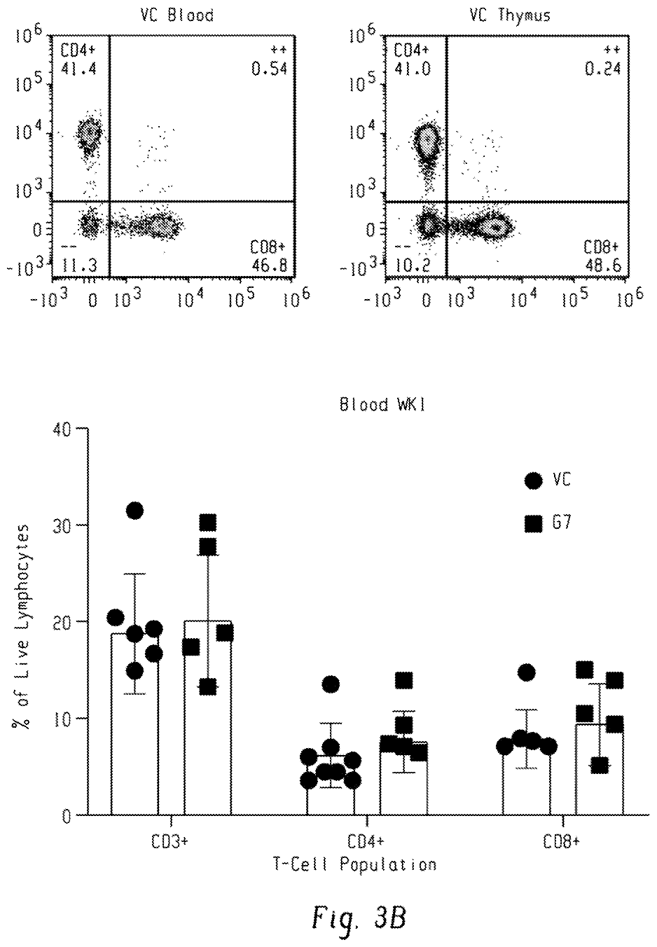

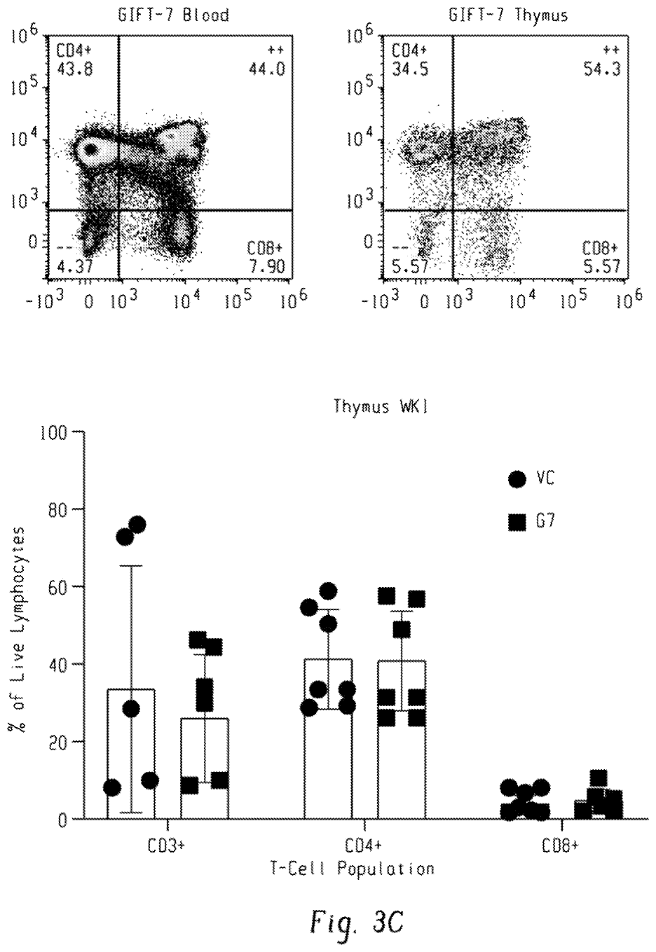

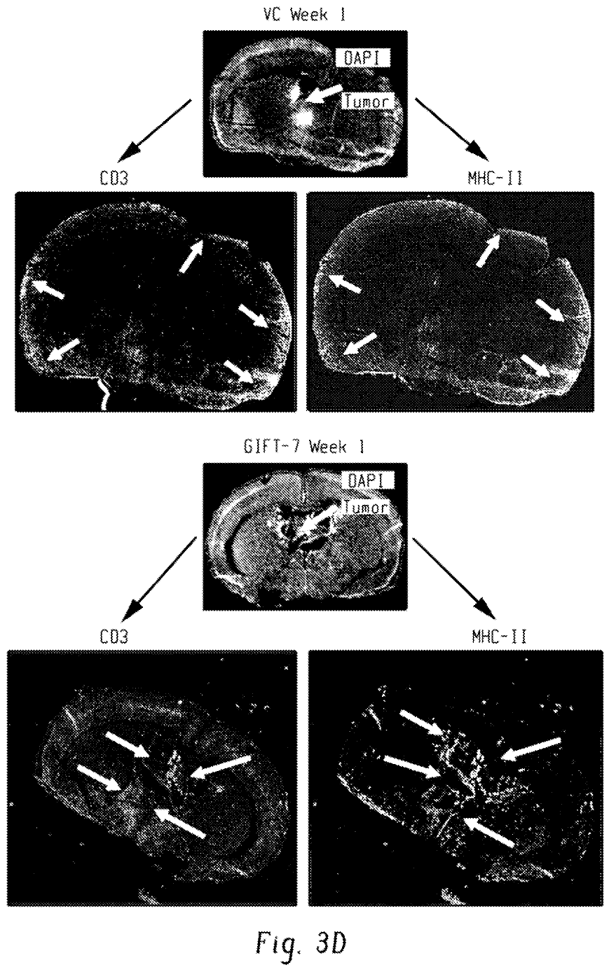

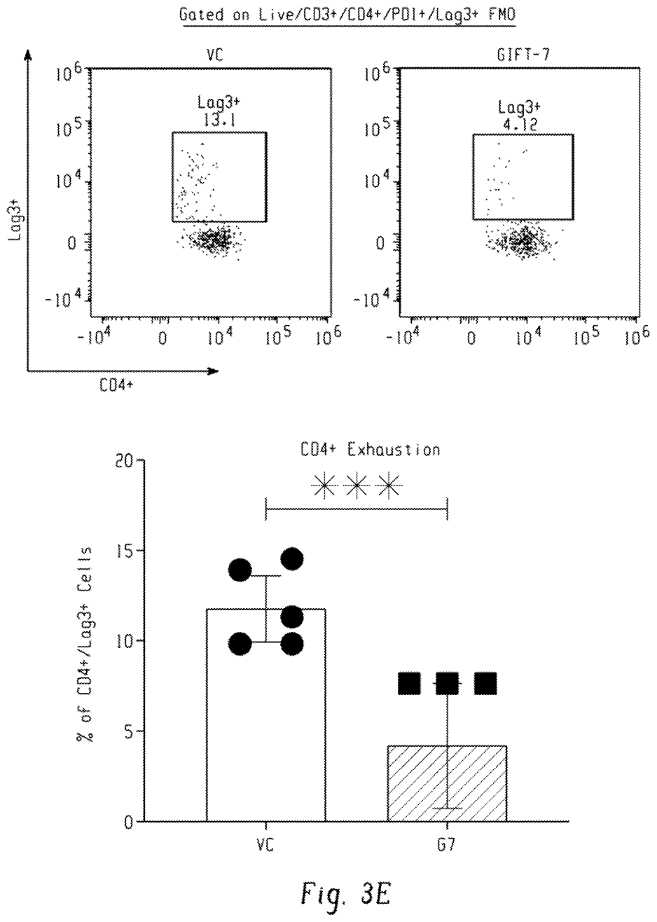

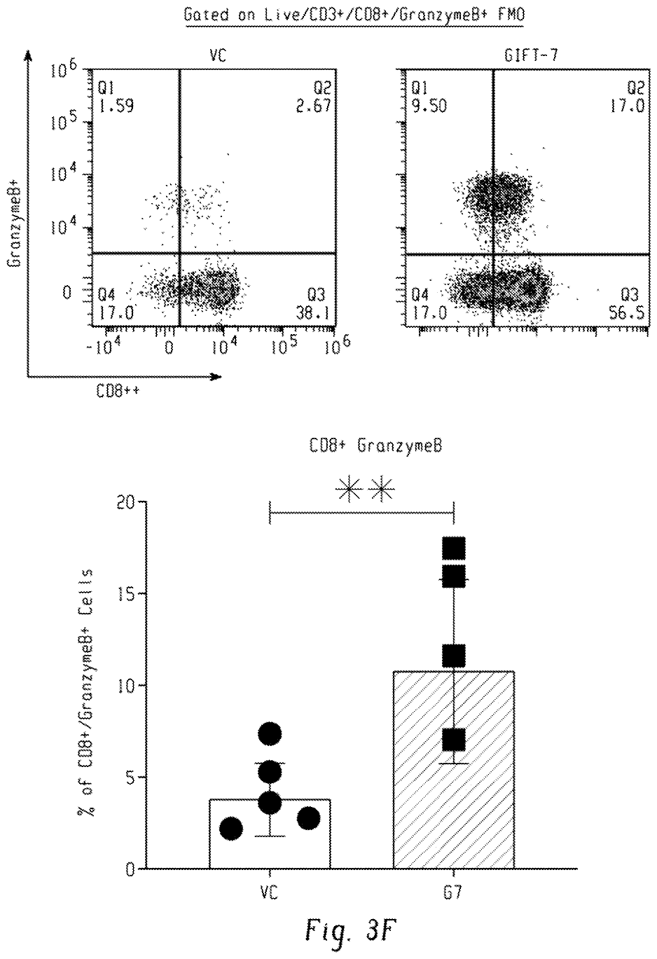

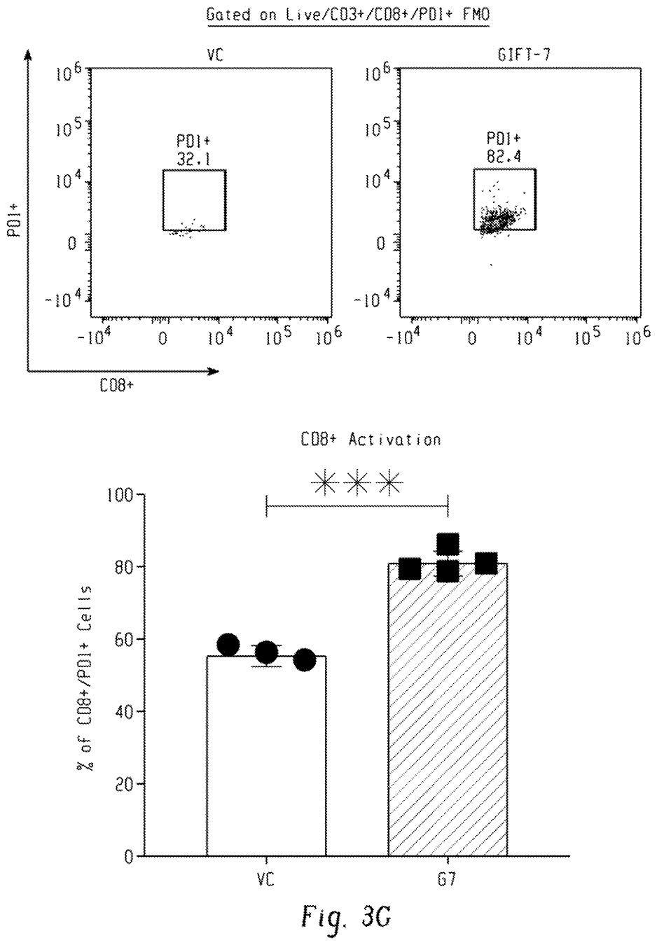

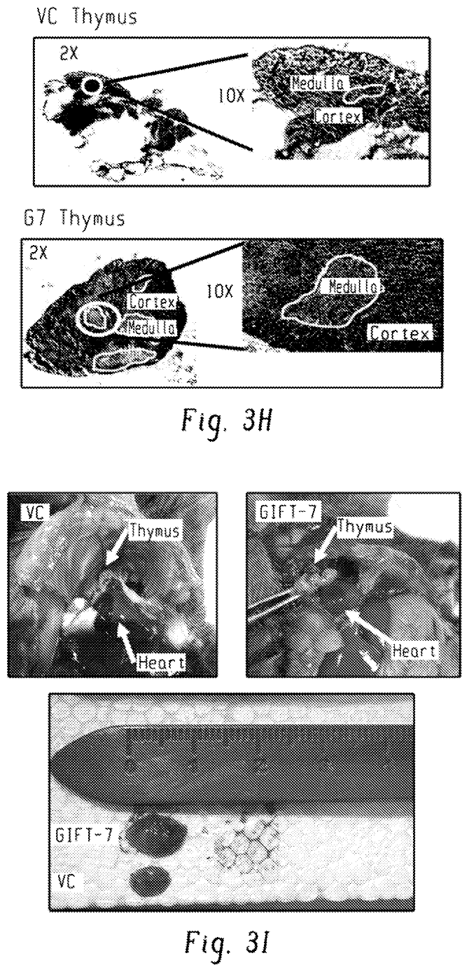

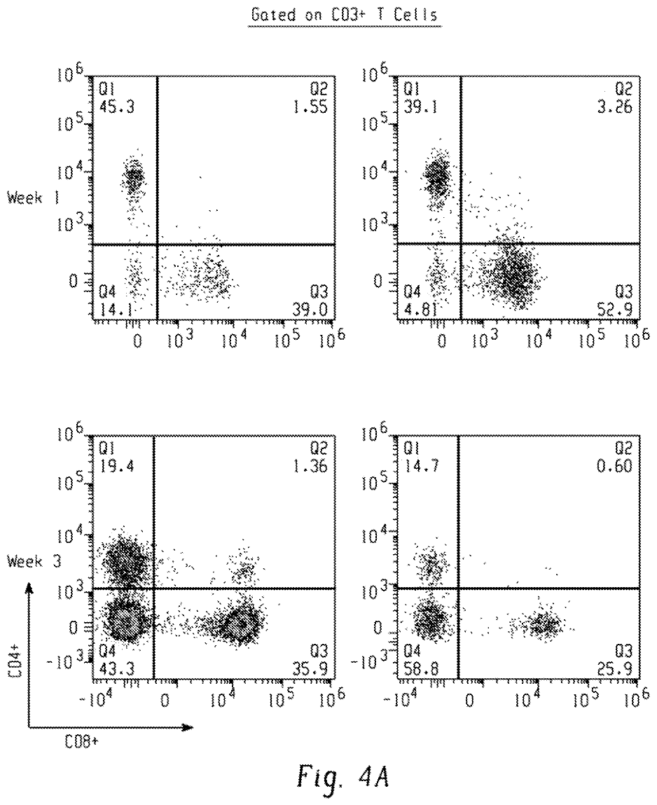

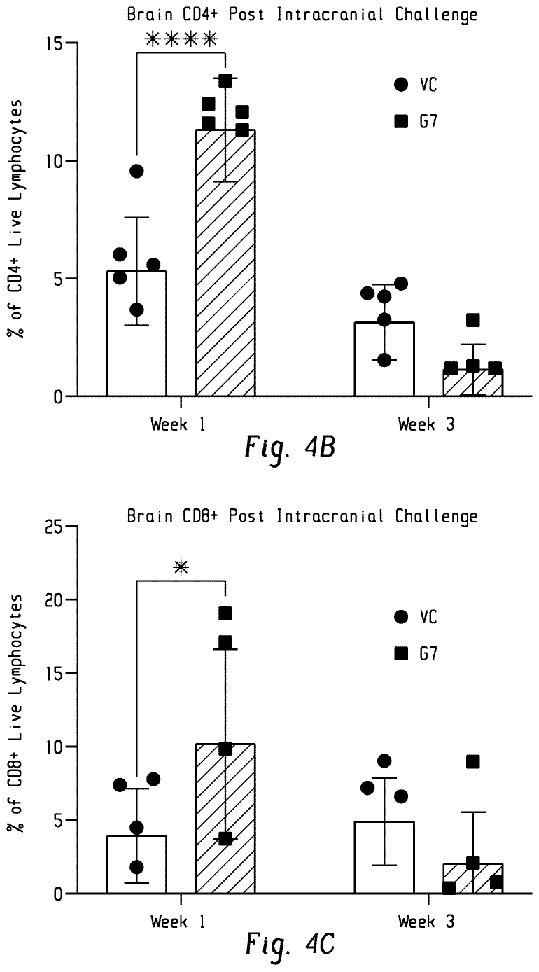

To understand the tumor immune microenvironment and immunologic mechanisms driving complete tumor clearance in the GL261 model, mice were sacrificed 1-week and 3-weeks after intracranial tumor implantation following vaccination, and the immune cell makeup of the blood, brain and thymus, was analyzed. At 1-week post intracranial tumor implantation there was a significant increase in absolute numbers of CD3+, CD4+ and CD8+ lymphocytes within the brains of mice from the GIFT-7Tvax group when compared to VC (VC vs GIFT-7Tvax: CD3+: 7.8% Vs 32.3% p<0.0001; CD4+: 2.8% Vs 9.1% p<0.0001; CD8+: 2.1% Vs 8.1% p<0.001) (FIG. 3A). This GIFT-7Tvax mediated T-cell increase was unique to the intracranial compartment, as it was not seen within either the peripheral blood or thymic compartments (Blood: VC vs GIFT-7Tvax: CD3+: 19% Vs 20.5% p=NS; CD4+: 6.4% Vs 7.8% p=NS; CD8+: 8.1% Vs 9.8% p=NS; Thymus: VC vs GIFT-7 CD3+: 34.6% Vs 26.6% p=NS; CD4+: 26.8% Vs 26.5% p=NS; CD8+: 4.3% Vs 4.8% p=NS) (FIGS. 3B and C). Immunofluorescence (IF) was utilized to visualize the increased intracranial recruitment of T-cells and demonstrated increased intratumoral CD3 and MHC-II staining exclusively in the GIFT-7Tvax group 1-week post intracranial tumor implantation (FIG. 3D). To understand the overall phenotype and functional status of the tumor infiltrating T-cells, exhaustion, cytotoxicity, and overall activation status was profiled using flow cytometry. Intracranial tumor infiltrating T191 cells in the GIFT7-Tvax group showed reduced CD4+ T-cell exhaustion (VC vs GIFT-7: CD3+/CD4+/PD1+/Lag3+: 11.8% Vs 4.4% p<0.001) (FIG. 3E), increased CD8+ cytotoxicity (VC vs GIFT-7: CD8+GranB+: 3.7% Vs 10.8% p<0.01) (FIG. 3F), and increased activation within the CD8+ compartment (VC vs GIFT-7: CD3+/CD8+/PD1+/Lag3-57.7% Vs 83.6% p<0.001) (FIG. 3G). In line with previous findings, a robust increase in the size of the thymus in the GIFT-7Tvax group was observed compared to VC at 1-week post intracranial tumor implantation. H&E staining showed an increase in the size of the thymic medullary compartments specifically in the GIFT-7Tvax mice (FIGS. 3H & I). Further examination revealed that the robust intracranial T-cell response seen at 1-week is lost by week 3 (Week-3 VC to GIFT-7: CD8+: 5.0% Vs 1.8% p=NS; CD4+: 3.2% Vs 1.2% p=NS) (FIGS. 4A, B and C). This suggests that the GIFT-7Tvax mediated systemic T-cell response is rapidly mobilized when the tumor is growing but is subsequently lost once the tumor is cleared from the intracranial compartment (roughly 3-weeks post implantation as suggested by FIG. 1G). These results demonstrate that GIFT-7Tvax regenerates the aging thymus and increases systemic T-cell circulation and trafficking to the brain, in response to the tumor.

### Example 3: Gift-7Tvax Results in Increased Systemic IL-1B and Formation of Hyperactive Dendritic Cells

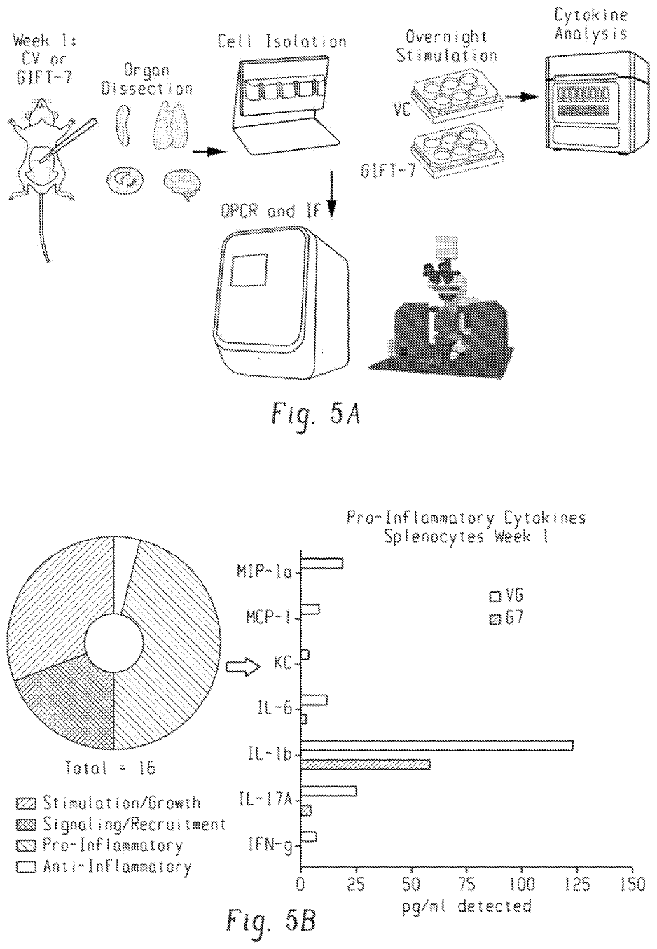

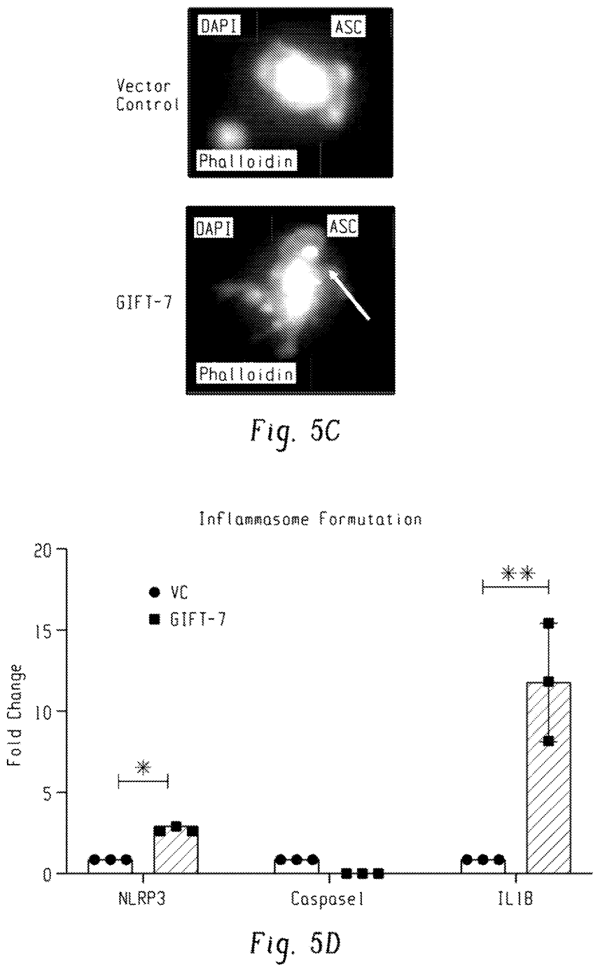

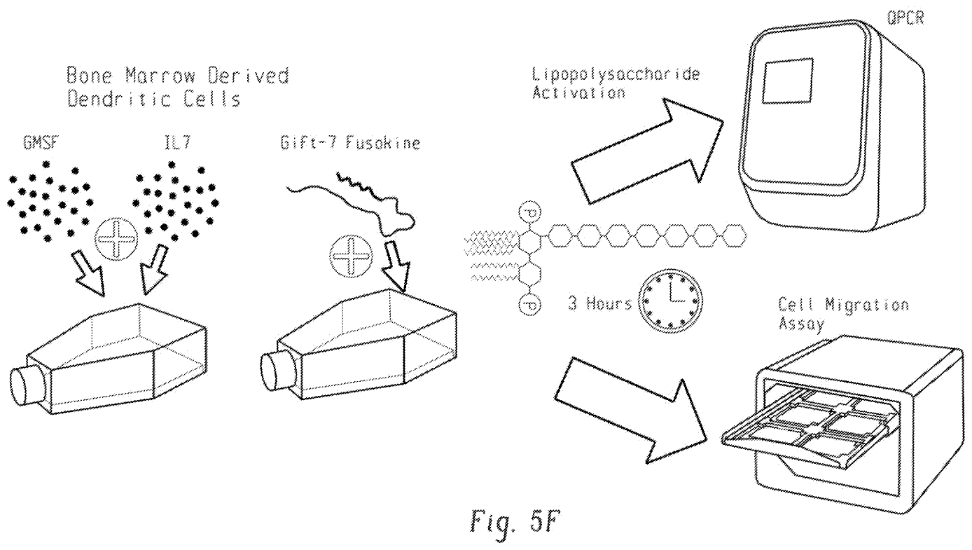

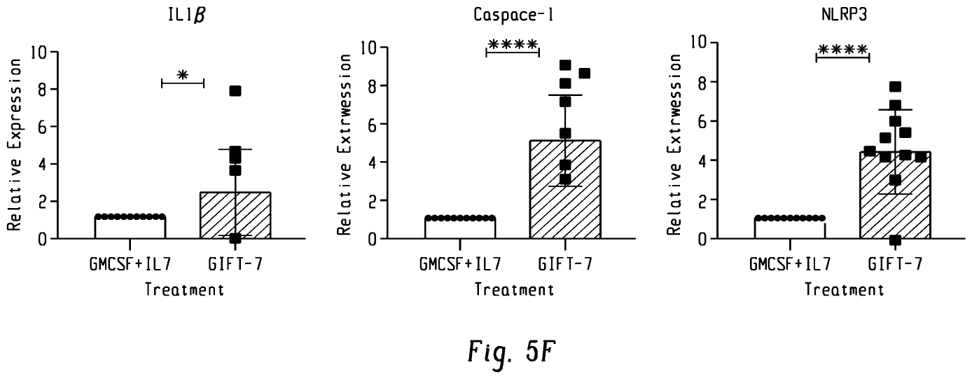

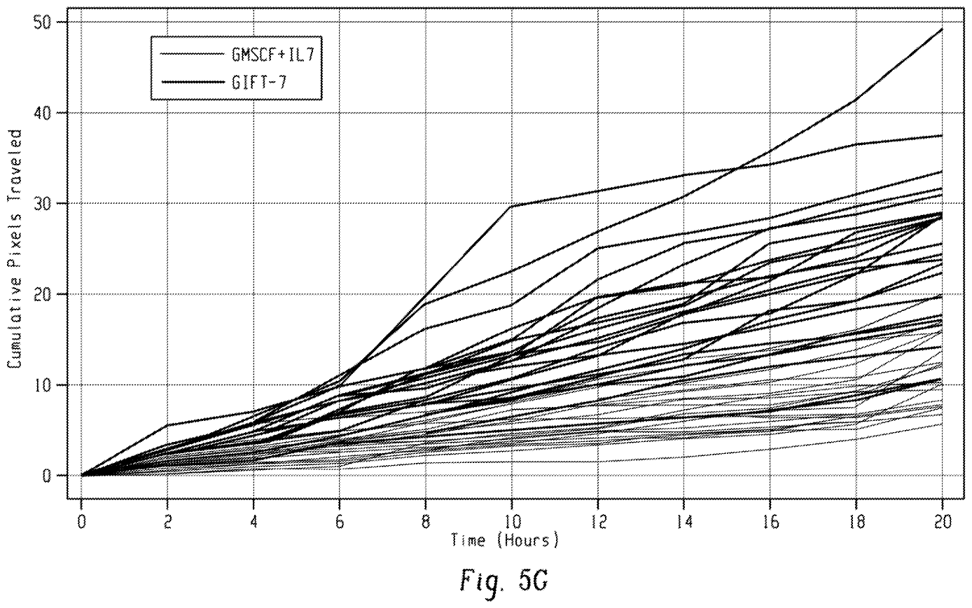

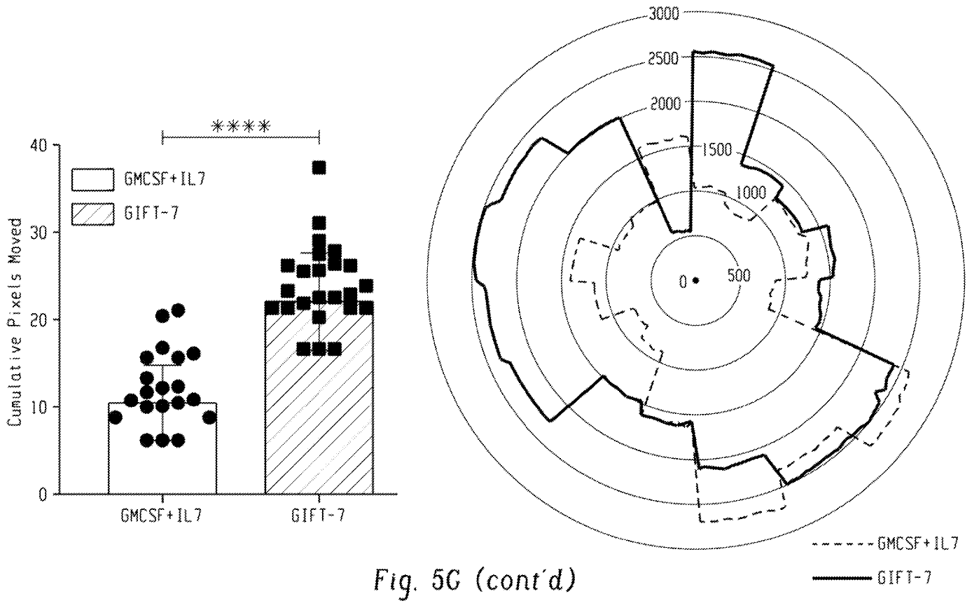

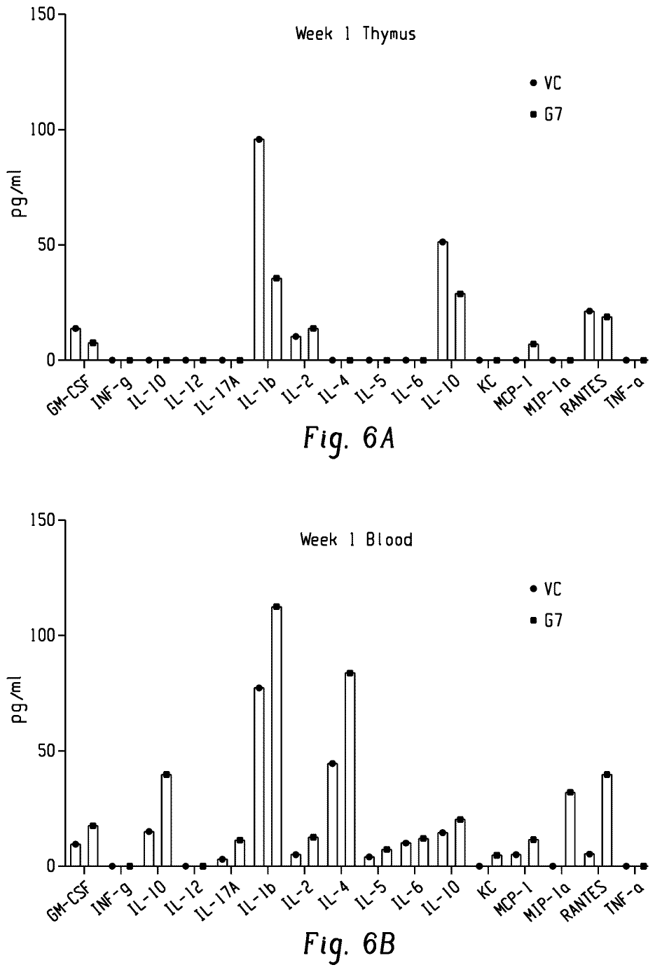

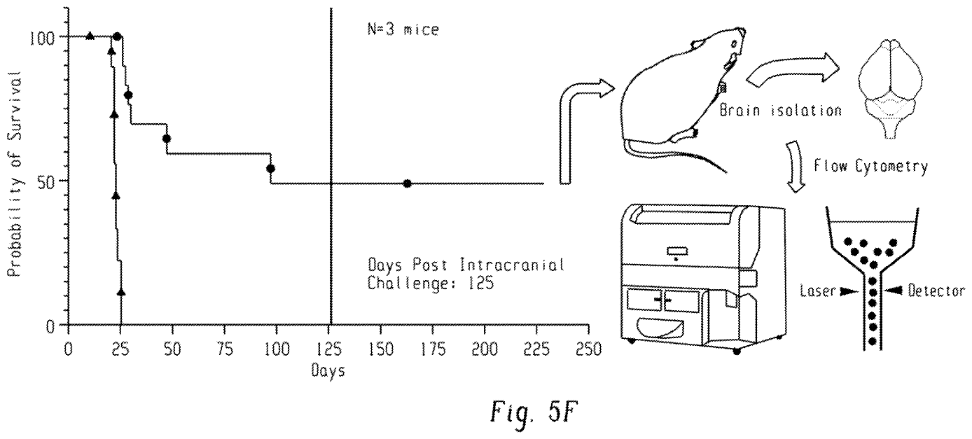

T-cell recruitment to a specific site relies on a complex network of cytokine signaling and secretion. To understand global cytokine secretion in the GIFT-7Tvax model, an unbiased cytokine screen was conducted across the blood, thymus, and spleen at 1-week post intracranial tumor implantation (FIG. 5A). The analysis of pro-inflammatory cytokines showed a significant increase in IL-Iβ in the GIFT-7Tvax mice (FIG. 5B, FIGS. 6 A and B). IL-1β is a pro-inflammatory cytokine that is produced by several cells within the innate immune system, such as macrophages and dendritic cells (DCs). It is first synthesized in a precursor form and cannot be activated until caspase mediated cleavage of the precursor takes place within the inflammasome. Recent studies have demonstrated that increased inflammasome assembly, and thus IL-1β secretion, can be caused by DC hyperactivation leading to more robust antigen presentation and T-cell recruitment. This effect has been shown to boost anti-tumor response and mediate long term-immunity in a mouse model of lymphoma and melanoma. To assay inflammasome formation, we isolated DC's from the spleens of mice 1-week post intracranial tumor implantation in both GIFT-7 Tvax and VC conditions. Adaptor molecule apoptosis-associated speck-like protein containing a CARD (ASC) speck formation staining, a reliable readout of inflammasome formation, showed inflammasome speck formation in the DC's from the GIFT-7TVax mice, while VC mice DC's displayed no speck formation (FIG. 5C). For a quantitative measurement, the same cells were subjected to QPCR for the genes present in the inflammasome signaling cascade. NLRP3 and IL-1β were significantly increased in the isolated DC's from the GIFT-7Tvax group when compared to VC (VC vs G7: IL-1β: 12.2-fold Vs 1-fold p<0.01; NLRP3: 2.12-fold Vs 1-fold p<0.05) (FIG. 5D). To confirm that the increased inflammasome formation was the result of the GIFT-7 fusokine mediating DC hyperactivation, bone marrow derived DCs (BMDC's) were subjected to in vitro stimulation with both recombinant IL-7 and GM-CSF (the components of the GIFT-7 fusokine) and the GIFT-7 fusokine itself (FIG. 5E). Inflammasome cascade markers IL-1β, Caspase-1, and NLRP3 were analyzed with QPCR on isolated BMDC's showing robust increases in all genes when stimulated with the GIFT-7 fusokine compared to its component parts (GMCSF+IL-7 vs GIFT-7: IL-1β: 4.5-fold Vs 1-fold p<0.05; Caspase1: 5.2-fold Vs 1-fold p<0.0001; NLRP3: 4.4-fold Vs 1-fold p<0.0001) (FIG. 5F). Along with increased inflammasome formation, hyperactive DCs are known to be more motile than native DC's, therefore, BMDC movement was visualized and quantified for 20 hours using an Incucyte incubator insert post stimulation with GIFT-7 or GMCSF+IL7. DCs stimulated with GIFT-7 traveled across more pixels and exhibited increased radial movements (quantified by Fiji) compared to GMCSF and IL-7 stimulated DCs (GIFT-7 vs GMCSF+IL-7:22.4 pixels Vs 10.7 pixels p<0.0001) (FIG. 5G). These data indicate that GIFT-7 exposure results in IL-1β mediated DC hyperactivation that contributes to the robust GIFT-7Tvax T-cell response.

### Example 4: Gift-7Tvax Induces ROR1T+ TH-17 Lineage Effector Memory T-Cells and Reduces T-Cell Exhaustion in Long-Term Survivor Mice

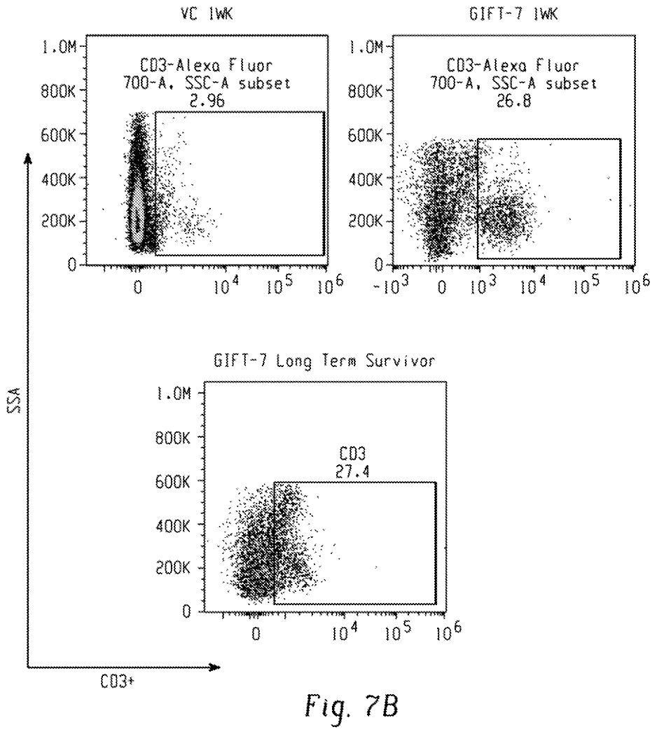

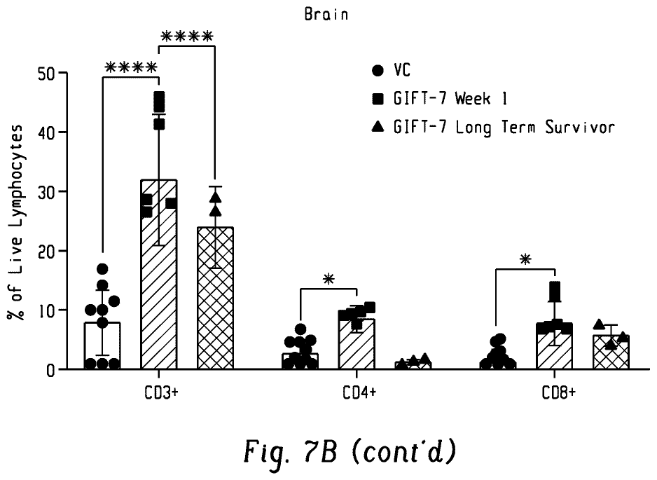

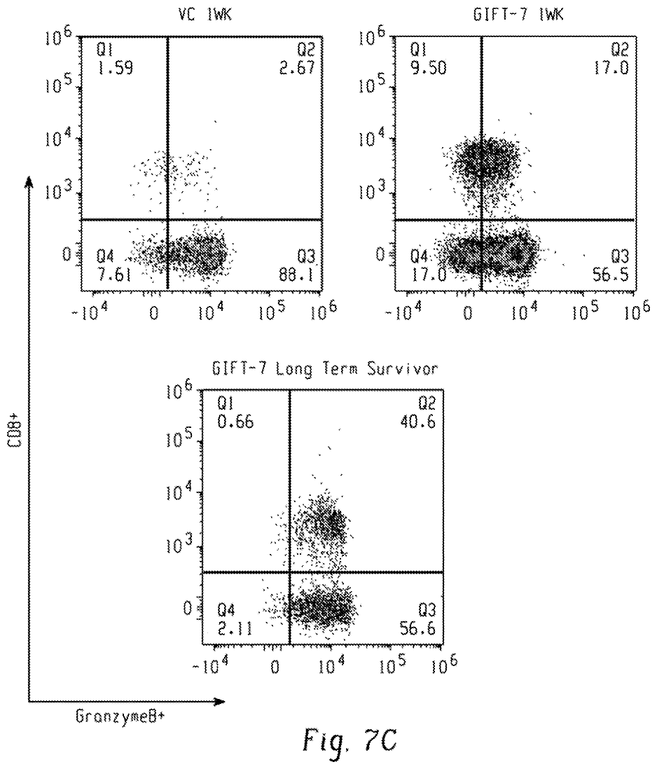

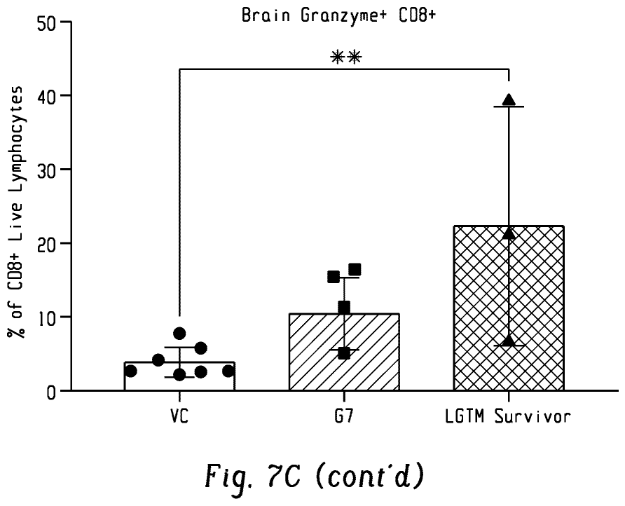

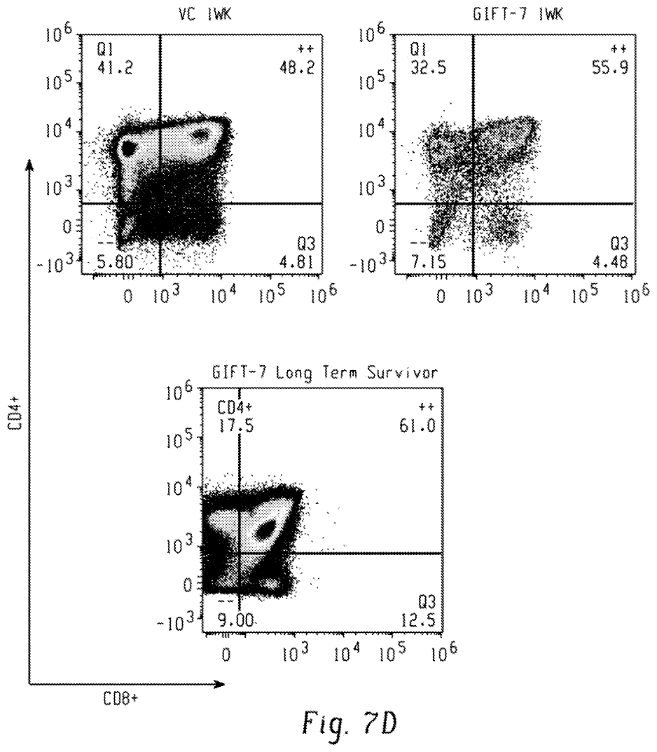

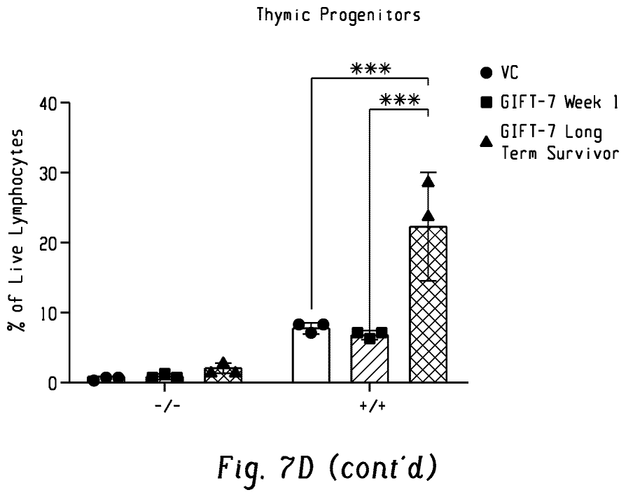

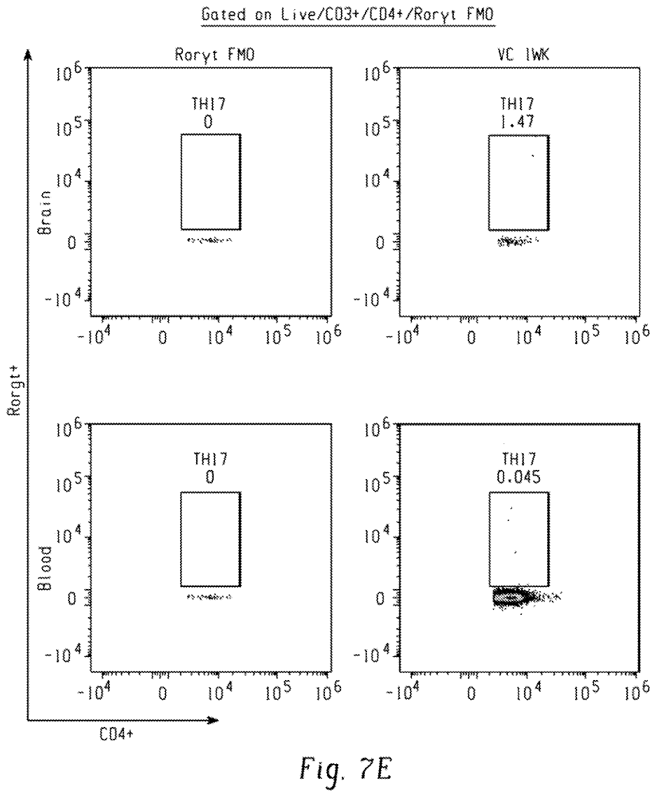

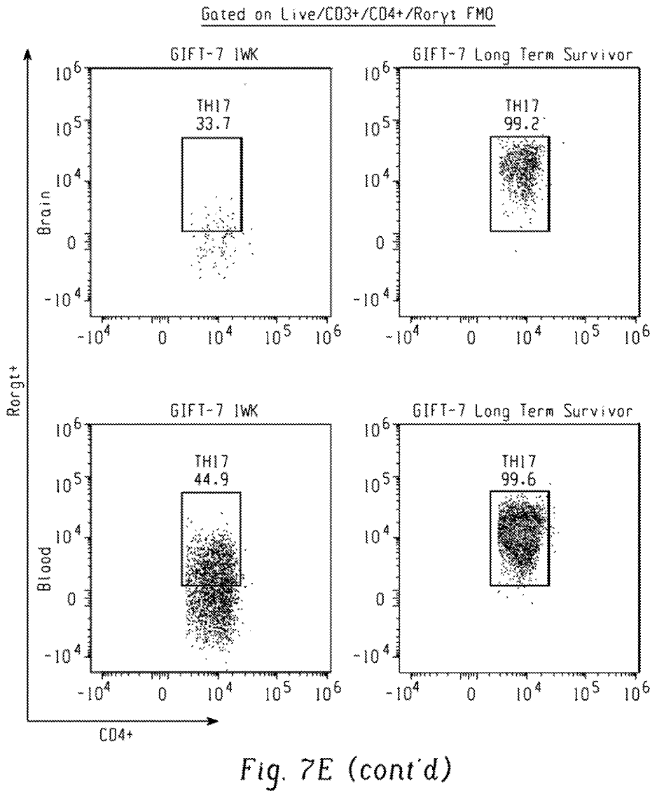

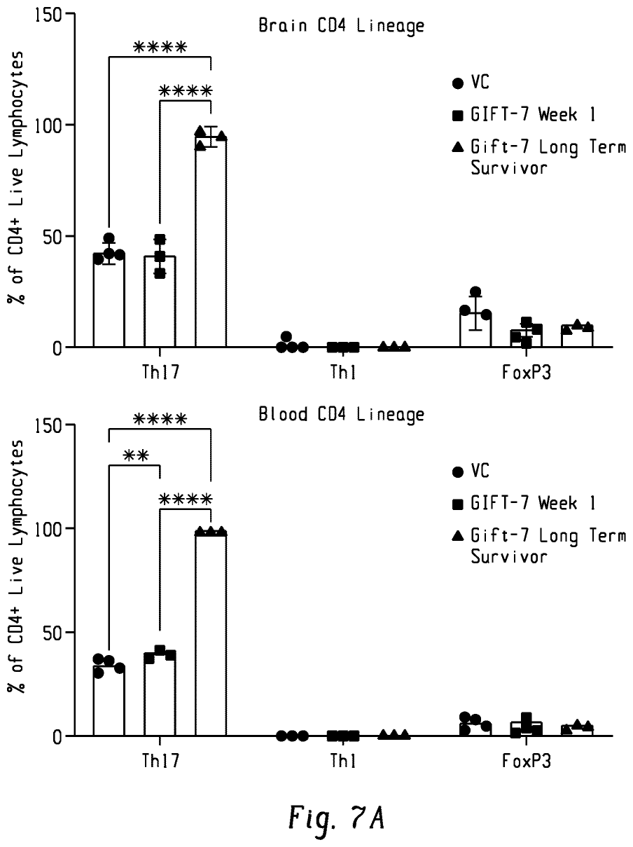

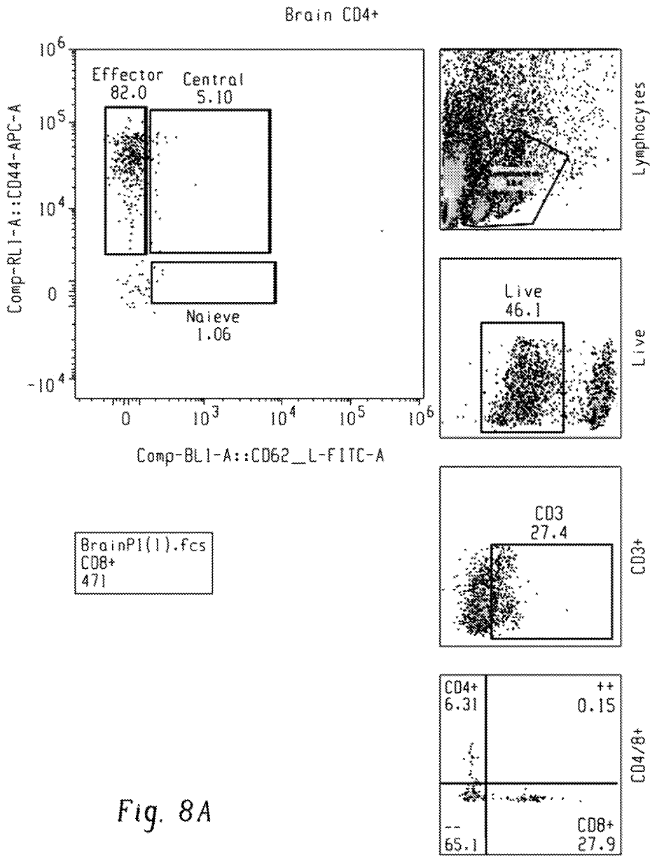

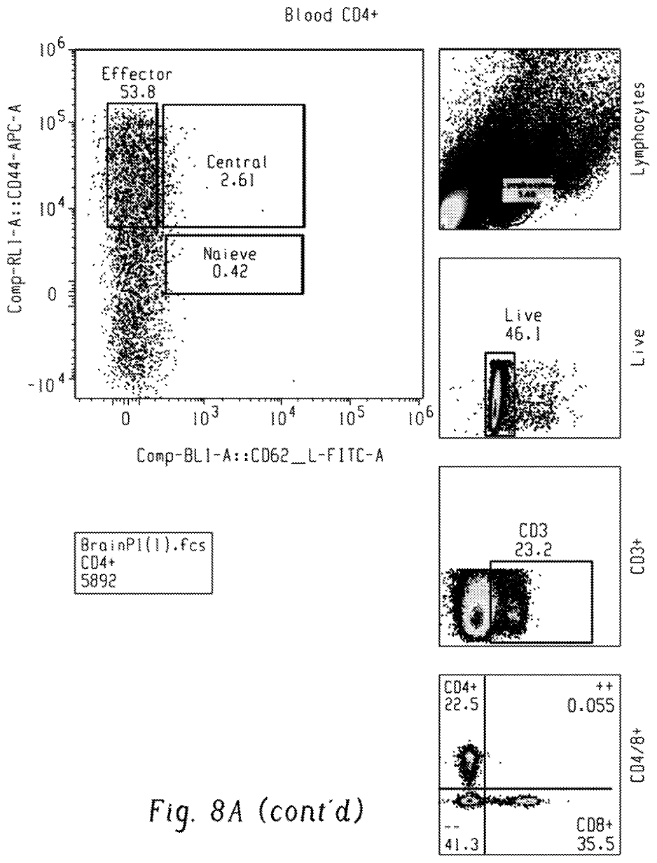

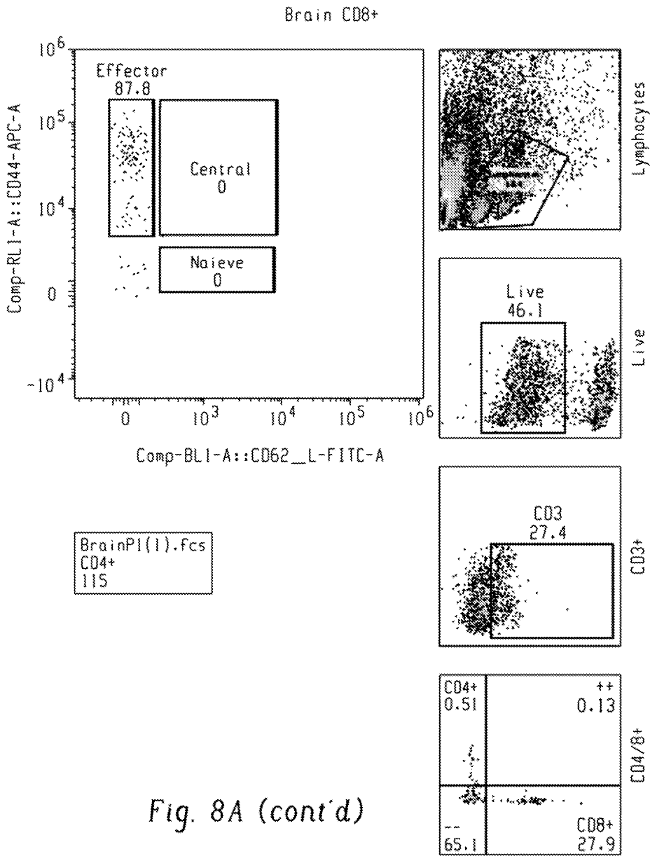

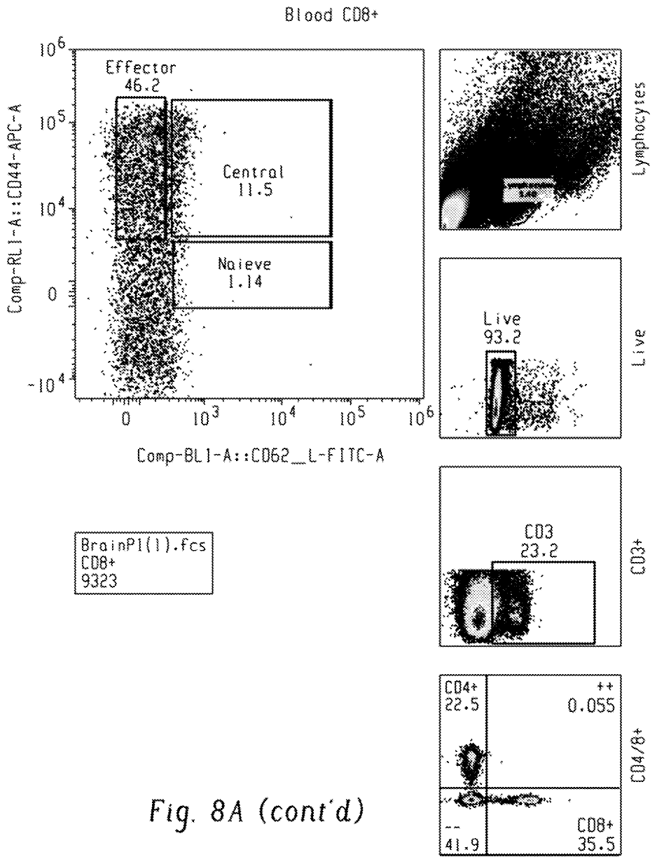

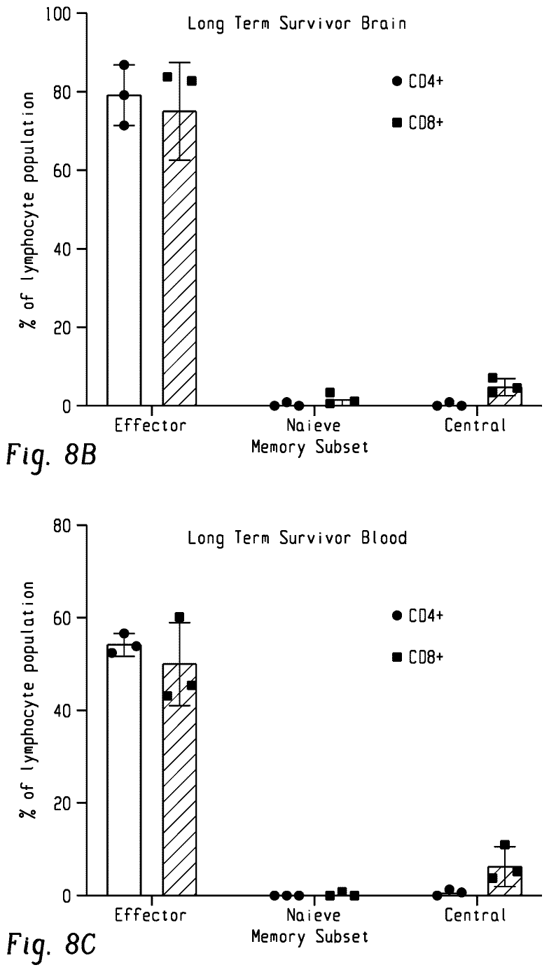

In the survival experiments, 50% of the mice from the GL261 GIFT-7TVax group completely cleared their intracranial tumor and became long-term survivors (LTS), which was defined as surviving 125 days post initial intracranial tumor implantation. At that time point the intracranial compartment was rechallenged by again implanting parental tumor cells on the contralateral side and monitored for OS for another 125 days. Interestingly, 100% of the LTS mice were able to completely reject the re-implanted tumor on the contralateral side (FIG. 7A). To understand the overall immune landscape of the LTS mice, the mice were sacrificed 250 days after initial intracranial tumor implantation and the T-cell compartment of the blood, brain, and thymus were profiled (FIG. 7A). LTS mice had increased levels of CD3+ T-cells within their brains when compared to VC control mice at 1-week post tumor implantation (LTS vs VC CD3+: 24.5% Vs 7.8% p<0.0001). However, they displayed slightly less CD3+, CD4+ and CD8+ T-cells in the brain compared to GIFT-7Tvax mice 1-week post tumor implantation, although a statistically significant difference was not reached (LTS vs GIFT-7TVax: CD3+: 24.5% Vs 32.3% p=NS; CD4+: 9.1% Vs 1.6% p=NS; CD8+: 8.1% Vs 5.7% p=NS) (FIG. 7B). CD8+ T-cells from LTS mice displayed increased GranzymeB levels when compared to VC mice (LTS vs VC: CD3+/CD8+/GranB+: 23.7% Vs 3.7% p<0.01) (FIG. 7C). Furthermore, LTS mice also had significantly increased numbers of CD4+/CD8+ thymic precursors, which have been shown to be the origin of both CD4+ helper and CD8+ cytotoxic T-cells, when compared to both VC and GIFT-7TVax mice at 1-week post intracranial tumor implantation (LTS vs VC: CD4+CD8+: 22.6% Vs 8% p<0.001; LTS vs GIFT-7TVax: CD4+CD8+: 22.6% Vs 7.3% p<0.001) (FIG. 7D). CD4+ memory subtypes, specifically Th-17 T cells, can become long lived effector memory cells and enhance durable immune response. It has also been observed that Th-17 cells migrate more effectively to the CNS parenchyma in autoimmune disorders such as multiple sclerosis, due to their secretion of IL-17 and IL-22 which can disrupt the tight junctions of the blood brain barrier (BBB). To understand if a T-cell lineage compartment shift was responsible for the durable anti-tumor response that had occurred in the LTS mice, the CD4+ memory compartment was assayed in both the brain and blood using FoxP3 (T-reg), Tbet (Th-1), and RORγt (Th-17) lineage markers. No statistically significant difference was observed between VC, GIFT-7TVax at 1-week and LTS mice across the Th-1 and T-reg lineages, however, a strong shift towards the Th-17 lineage was observed, in both the brain and blood of LTS mice, compared to either GIFT-7TVax at 1-week or VC (Blood: VC vs LTS: Th-17:33.4% Vs 99.4% p<0.0001; GIFT-7TVax vs LTS: Th-17:42.7% Vs 99.4% p<0.0001) (Brain: VC vs LTS: Th-17:41.8% Vs 95.3% p<0.0001; GIFT-7TVax vs LTS Th-17:41.9% Vs 95.3% p<0.0001) (FIGS. 7E and F). There was also a strong maintenance of the effector memory subtype in both the CD4+ and CD8+ lineages across the blood and brain of LTS mice (Sup FIGS. 8A, B and C). This data indicates that a GIFT-7TVax mediated CD4+ T-cell lineage polarization towards the Th-17 effector memory phenotype promotes intracranial tumor clearance and generates long-term immunity against GBM.

### Example: 5 Gift-7TVAX Remodels the T-Cell Receptor (TCR) Landscape by Increasing Overall TCR Repertoire Clonality During Early Anti-Tumor Response

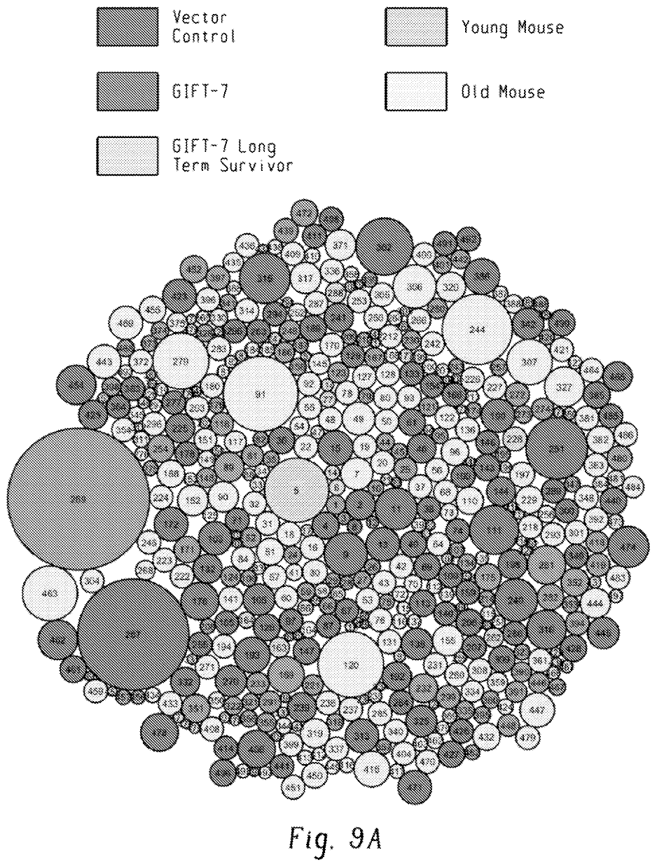

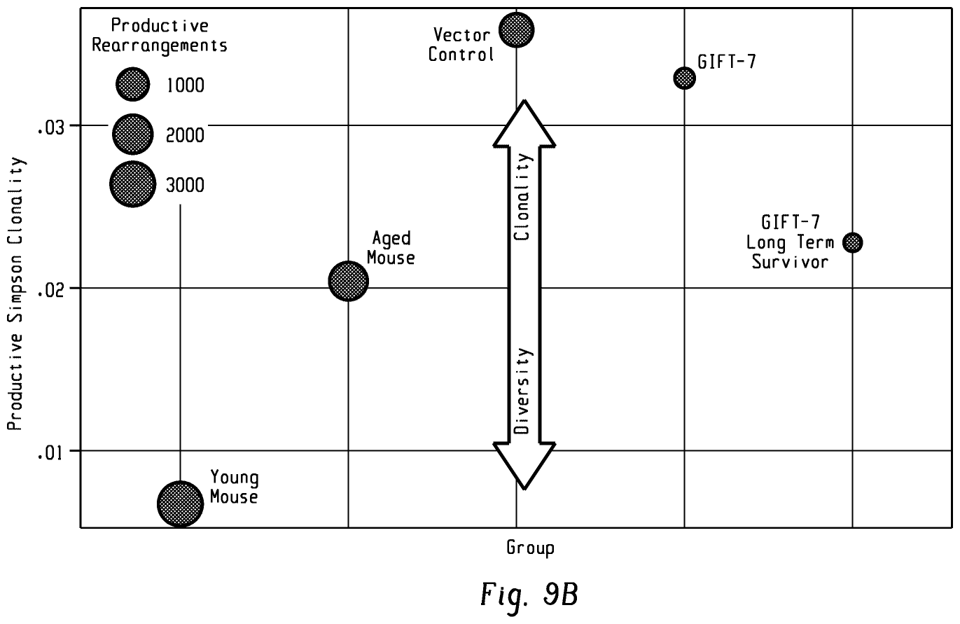

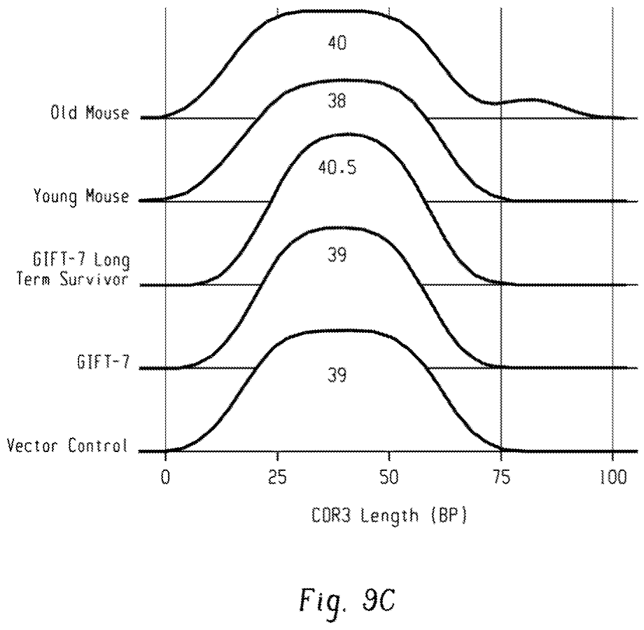

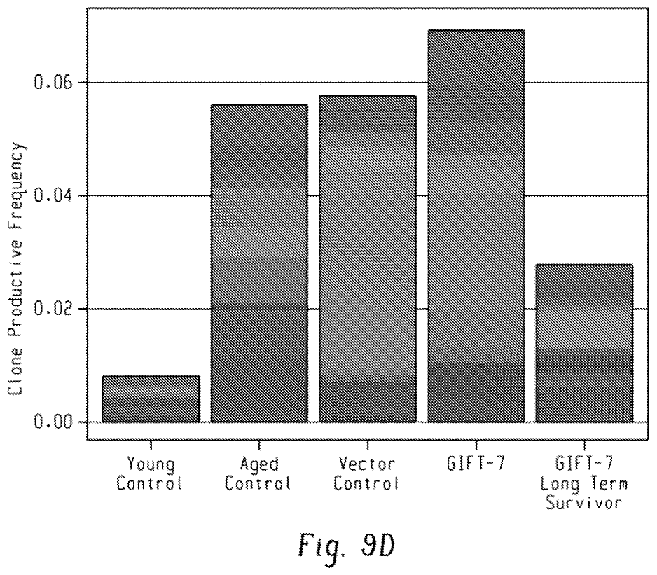

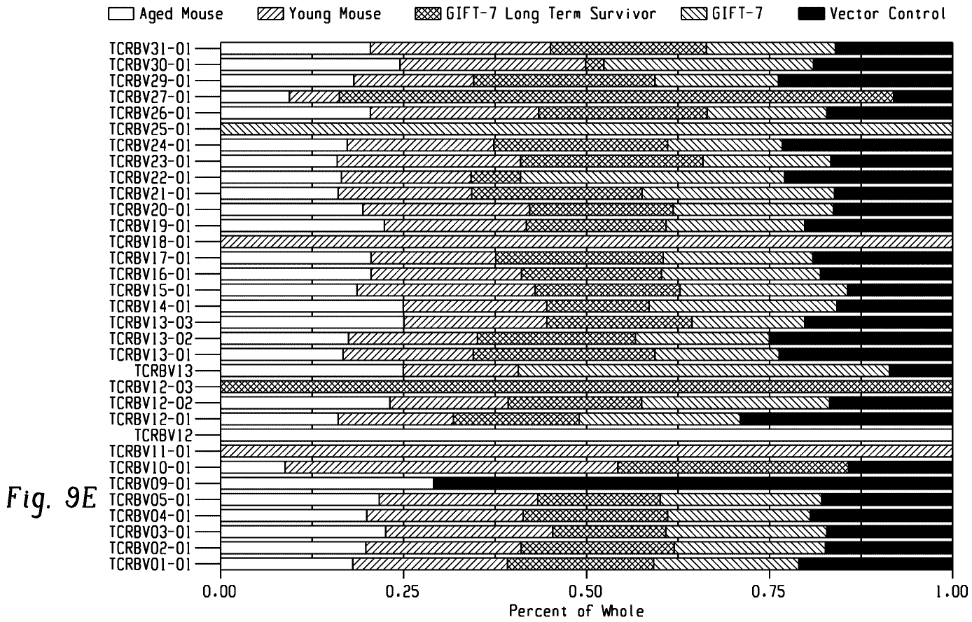

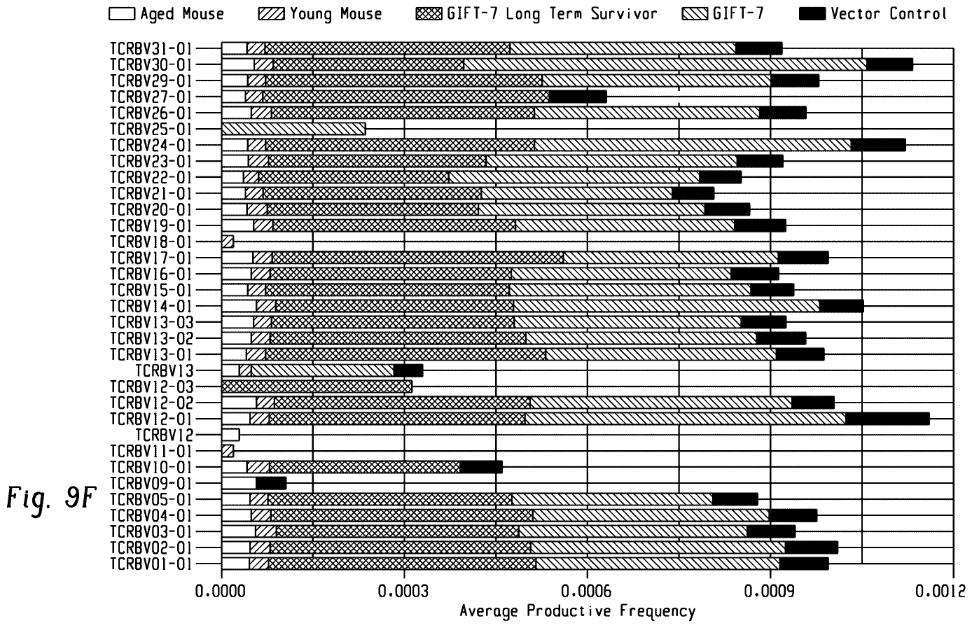

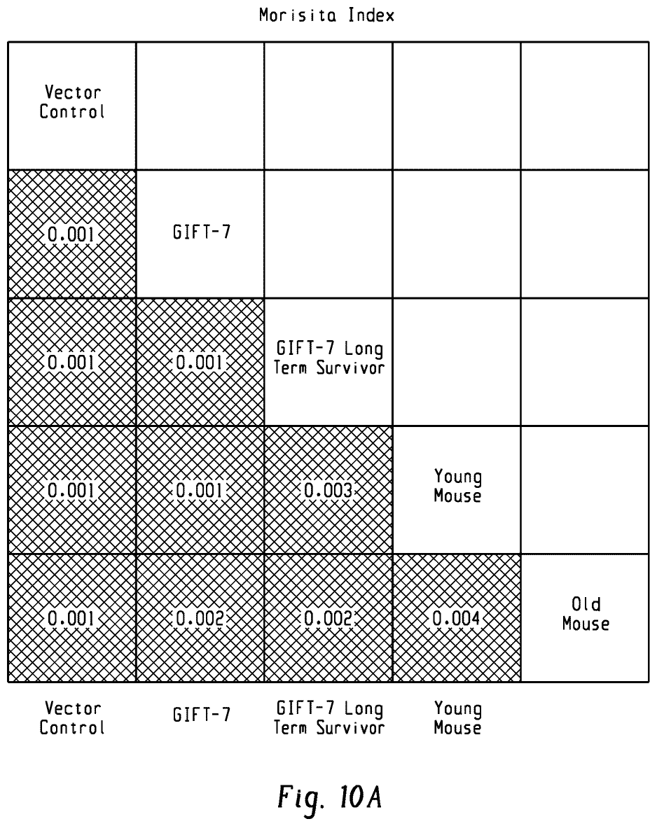

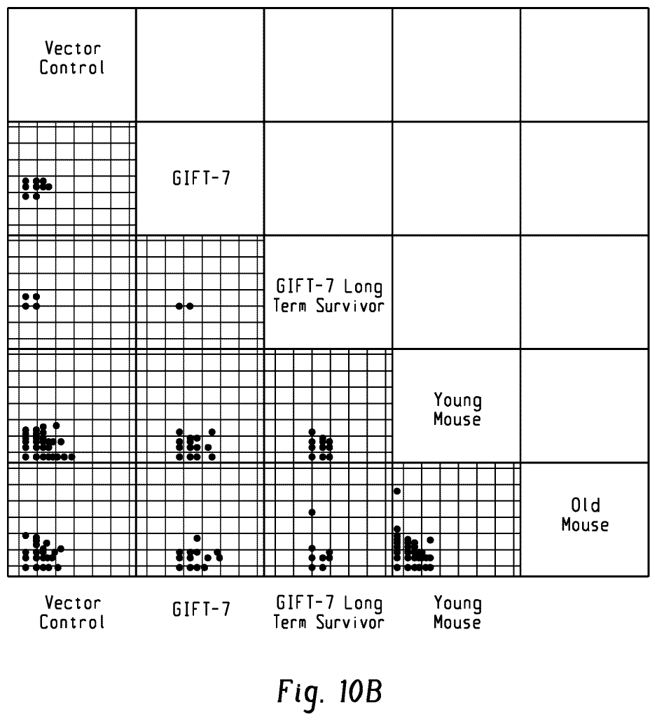

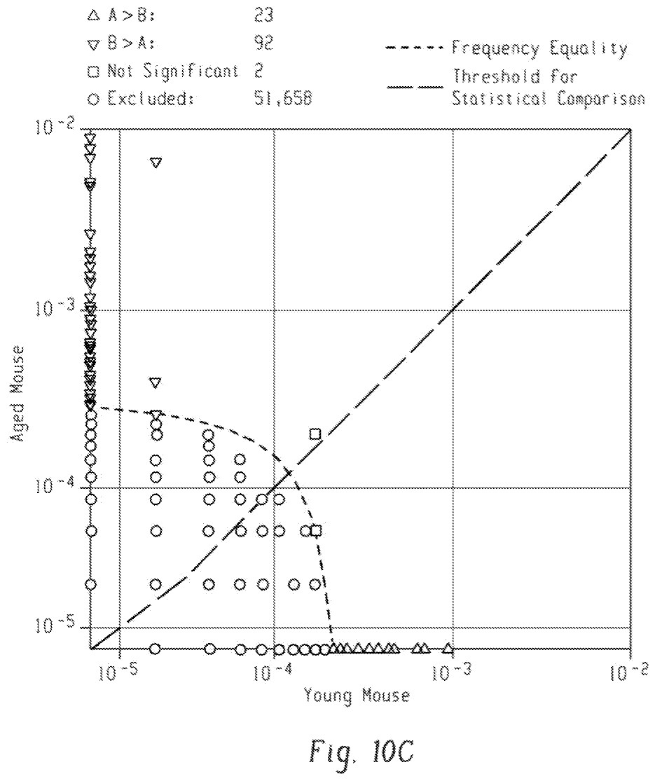

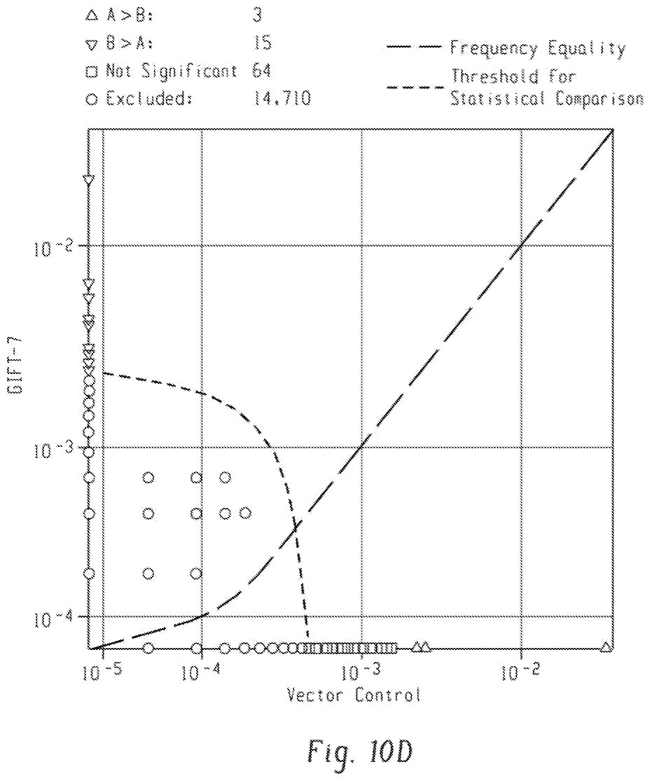

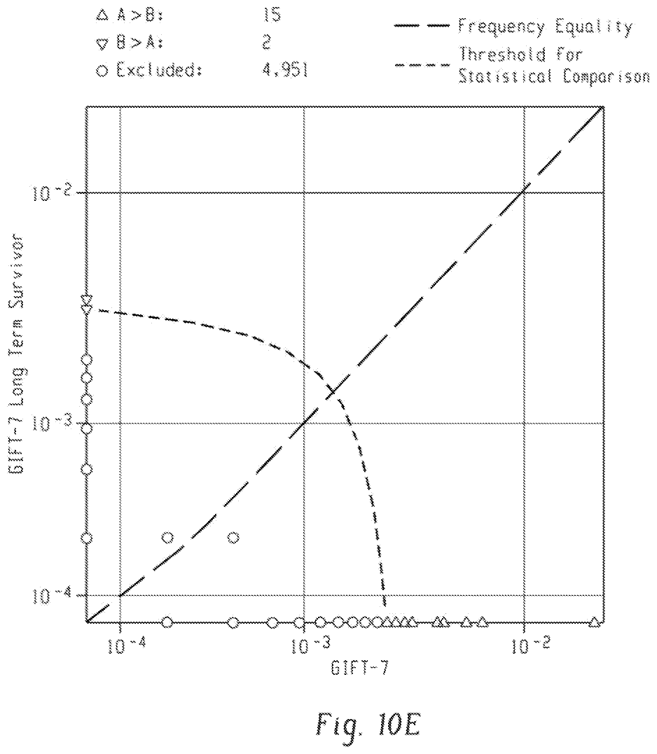

Since GIFT-7TVax regenerated the thymus, the primary organ for T-cell formation and antigen education, and increased intracranial T-cell presence was only seen in the GIFT-7TVax group, it was hypothesized that GIFT-7 preferentially generated/expanded tumor associated T-cells. To test this hypothesis, we analyzed the TCR landscape of the GIFT-7TVax mice during initial anti-tumor immune response, at 1-week post intracranial tumor implantation, as well as LTS mice with durable anti-tumor immunity, and compared them with young, aged, and VC mice. We performed bulk TCR sequencing using isolated genomic DNA from peripheral blood. Several different measures including rearrangement productive frequency, CRD3 length, clonal commonality, and TCRβ gene usage, were utilized across the groups to analyze repertoire diversity and composition. Global visualization of repertoire composition indicated expansion of dominant clones and repertoire focusing within the tumor bearing mice both the VC and GIFT-7TVax groups at the onset of the anti-tumor immune response, demonstrated by clone #267 (GIFT-7TVax) and #269 (VC) (FIG. 9A). LTS mice, which represent a longer term durable immunity against tumor, displayed less repertoire focusing and less dominating clones, similar to both the young and aged non-tumor bearing mice (FIG. 9A). Simpson clonality, which approaches a value of 1 if a repertoire is entirely monoclonal and a value of zero if a repertoire is completely unique, was utilized to understand the composition of the T-cell repertoires among our analyzed groups. In general, the repertoires within the groups were quite diverse and showed little similarity to one another when compared with Morisita Index (FIGS. 10A and B). A significant increase in the clonality of repertoires was observed in the tumor vaccinated groups (VC or GIFT-7TVax) when compared to the young non-tumor bearing mice (Dunn multiple comparison correction: VC to Young Control: . 024 Vs.006 p<0.05; GIFT-7TVax to young control: 0.018 Vs 0.006 p<0.05) (FIG. 9B). Additionally, GIFT-7Tvax at 1-week and LTS mice specifically showed a large decrease in the number of total productive β-rearrangements present indicating a possible shift away from the αβ phenotype and towards the γδ phenotype73,74 (FIG. 9B). γδT-cells have been shown to support the development of CD4+ Th-17 cells, which are enriched in the LTS mice (FIG. 9F), through the secretion of specific cytokines such as RANTES, MCP-1, IL17 and IP-10. Global cytokine analysis for γδ and Th-17 related cytokines showed increase in each of the above cytokines in GIFT-7TVax mice compared to VC (VC Vs GIFT-7TVax RANTES: 11.09 Vs 67.15; MCP-1:0 Vs 8.07; IL17: 4.60 Vs 24.91; IP-10:20.33 Vs 125.14) (FIG. 10C). There was little difference in the mean CDR3 transcript length between each of our groups, and while the aged non-tumor bearing mice did exhibit larger standard deviation in CDR3 transcript lengths relative to the other groups, statistical significance was not reached (standard deviation age mouse: 17.39 vs young mouse: 13.76, LTS: 10.81, VC: 13.42, G7: 11.68; F-test=NS) (FIG. 9C). Clone productive frequency for the top 20 clones was similar across the aged control, VC, and GIFT-7Tvax mice, and significantly lower in the young control mice while moderately lower in the LTS mice (GIFT-7TVax Vs Young Mouse: 0.003 Vs 0.0004 p<0.05, G7 vs LTS 0.003 Vs 0.002 p=NS) (FIG. 9D). Finally, TCRβ gene usage remained roughly proportionally consistent between groups (FIG. 9E), however, when productive frequency of the gene usage was measured both GIFT-7Tvax at 1-week and LTS mice showed higher overall TCRβ gene usage (VC Vs GIFT-7TVax: 0.0007 Vs 0.003 p<0.00001; VC Vs LTS: 0.0007 Vs 0.004 p<0.0001; G7 Vs LTS 0.003 Vs 0.004 p=NS) (FIG. 9F). Interestingly, unique TCRB gene usage was displayed in both the GIFT-7TVax group (TCRBV25-01) as well the LTS group (TCRBV12-03), indicating a likely tumor related clonal expansion. Individual clone tracking between groups also indicated little overall similarity with the highest frequency of shared clones occurring between the non-tumor bearing young and aged mice (Morisita Index 0.004, similar clones: 115) (FIGS. 10D, E, and F). Aging itself has been shown to have a significant impact on the TCR repertoire and in concordance with other groups studying age effects on the TCR repertoire, a difference in T-cell repertoire makeup was found between the young and aged non-tumor bearing mice. Young mice exhibited more repertoire diversity and less total clone productive frequency per rearrangement (FIGS. 9B and D), as well as a differential usage of TCRB genes both proportionally and in productive frequency (FIGS. 9E and F). These data show the clonal focusing of the TCR repertoires of vaccinated mice which relaxes somewhat as the tumor is cleared and homeostasis is re-achieved, retaining a possible γδ mediated durable Th-17 memory for long-term tumor specific immunological memory. Furthermore, the stark difference between the repertoires of aged and young mice independent of any experimentation is clear and most likely plays a role in better anti-tumor response on vaccination seen in the young mice (FIG. 2D).

### Example 6: GIFT-7TVAX is Therapeutic and Increases Overall Survival in a Clinically Relevant Aged Mice Model of Glioma

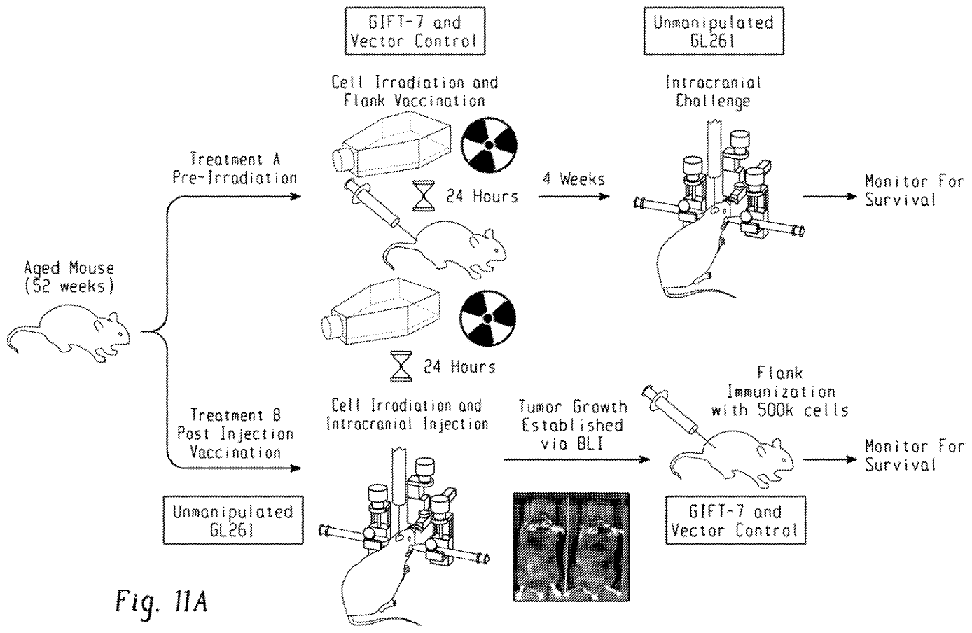

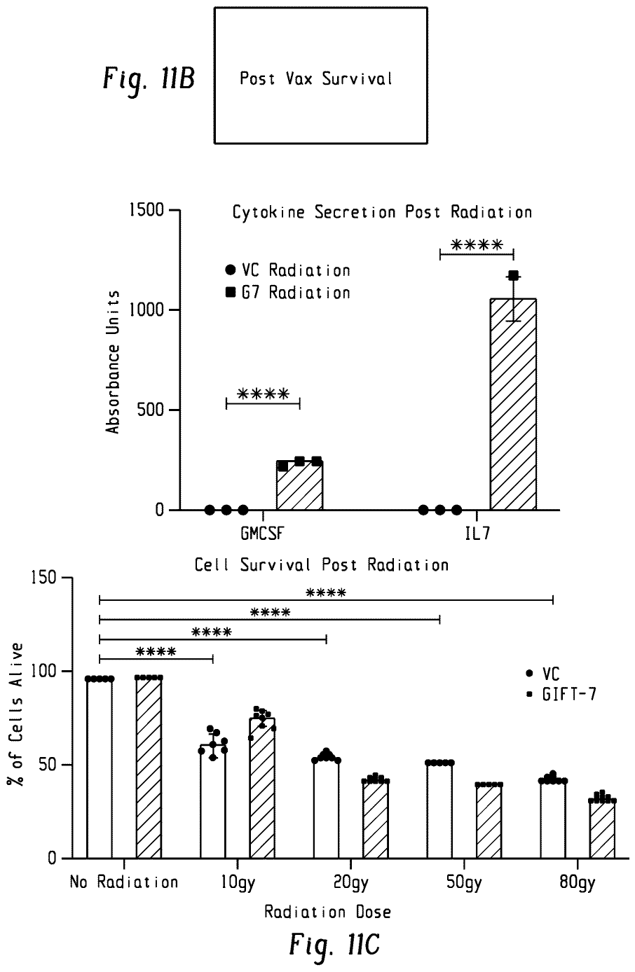

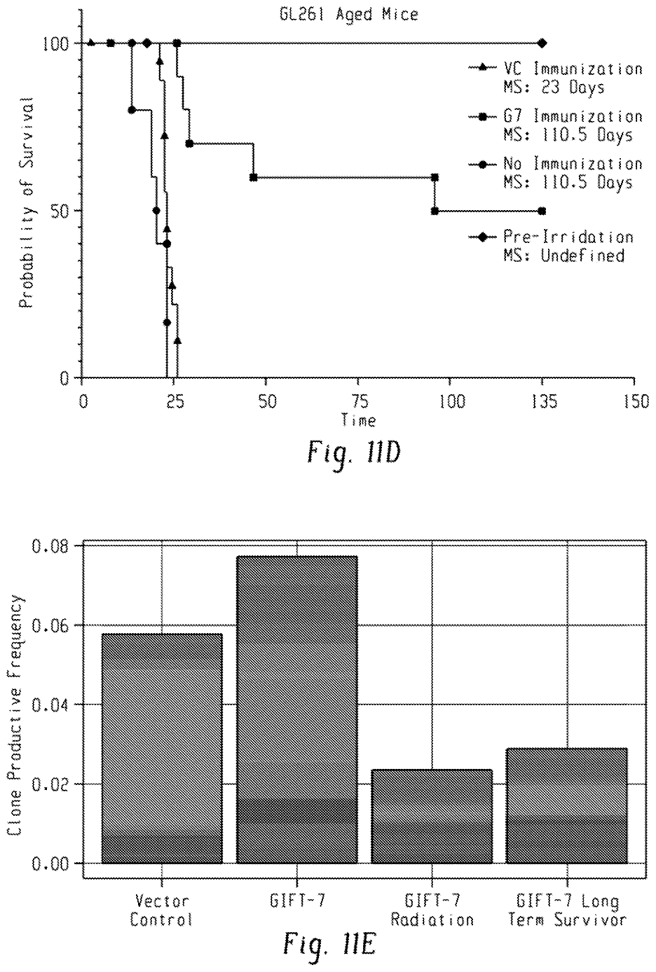

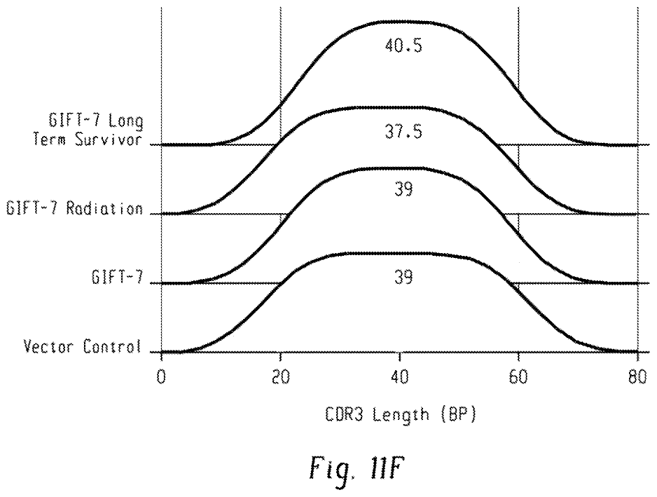

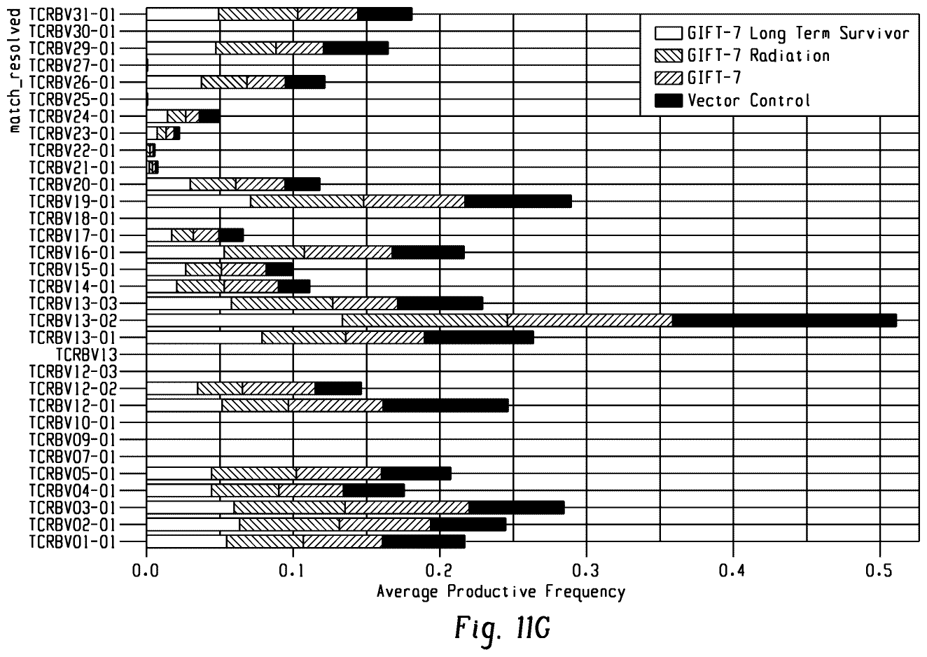

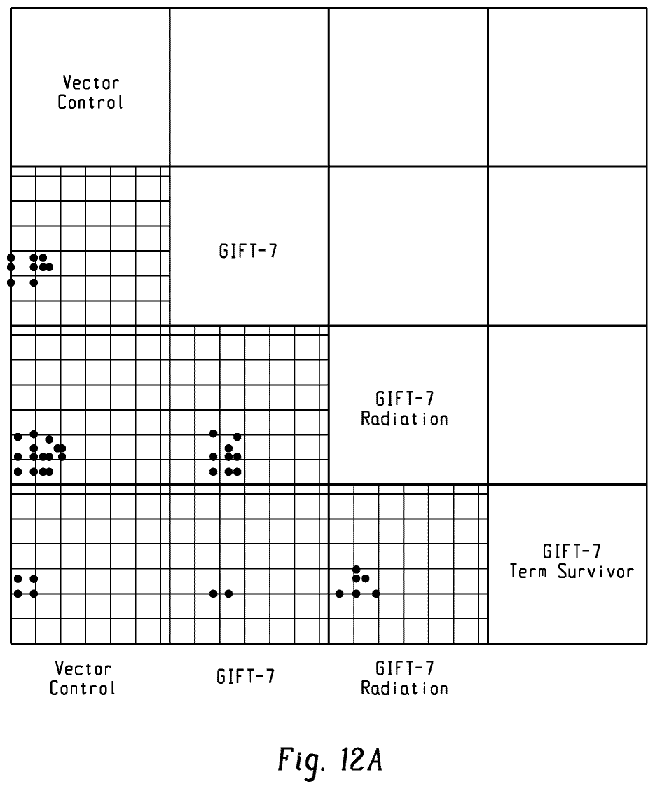

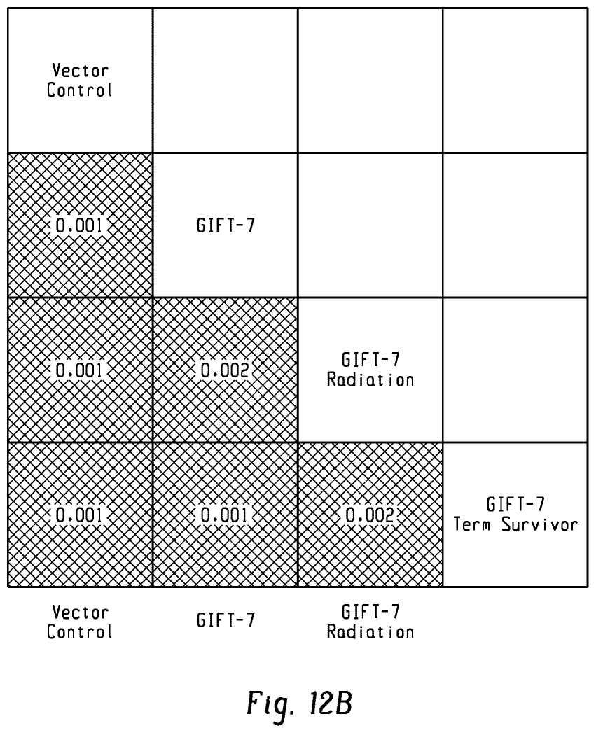

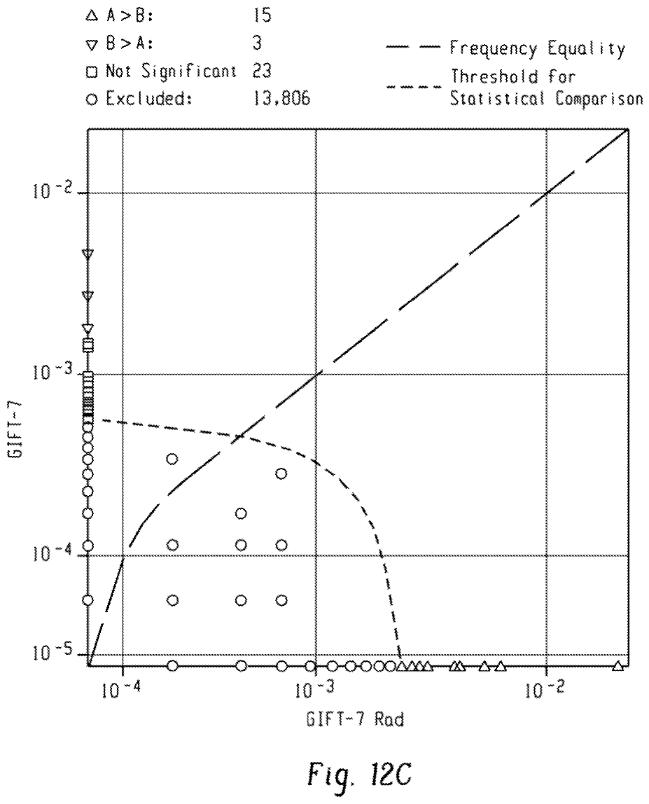

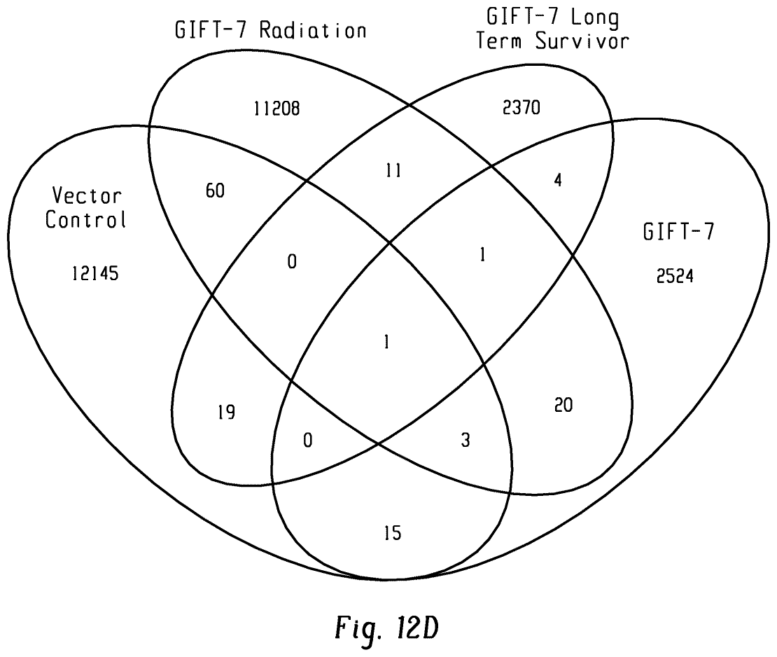

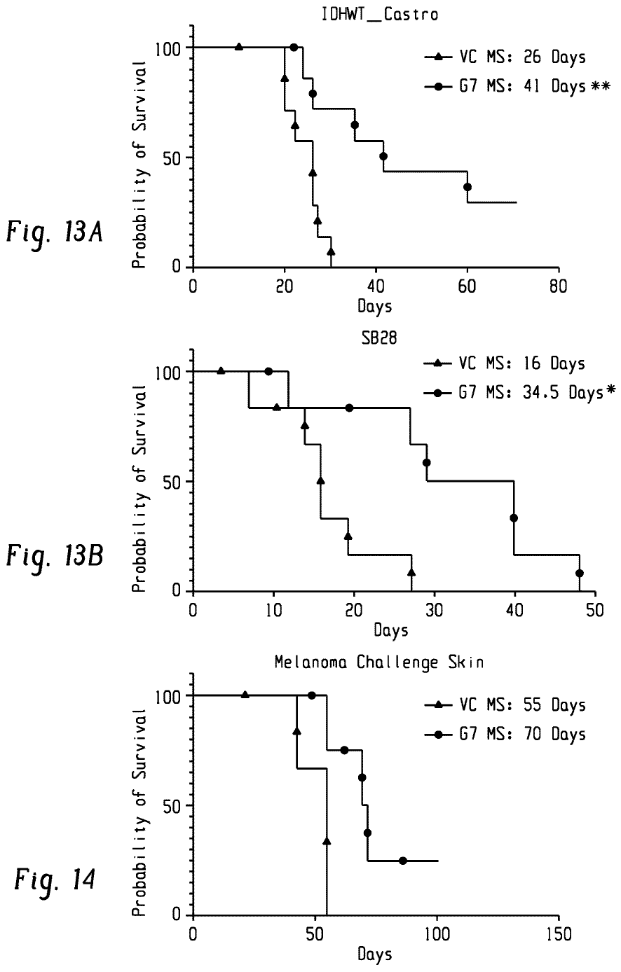

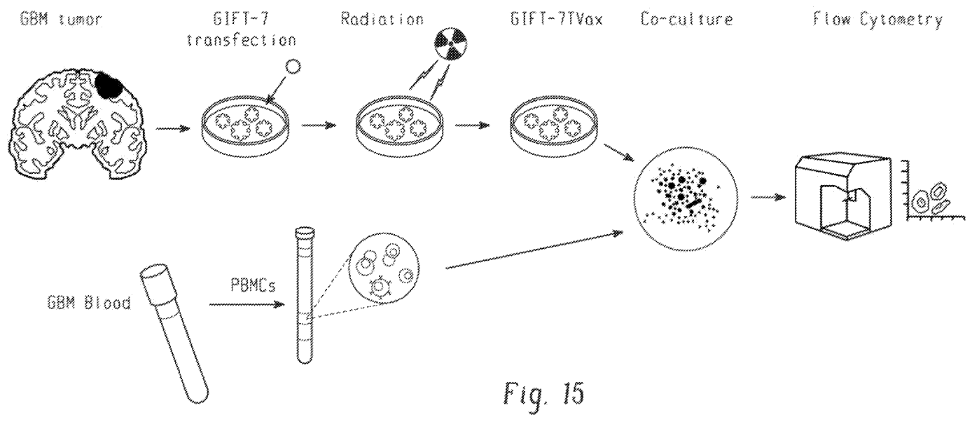

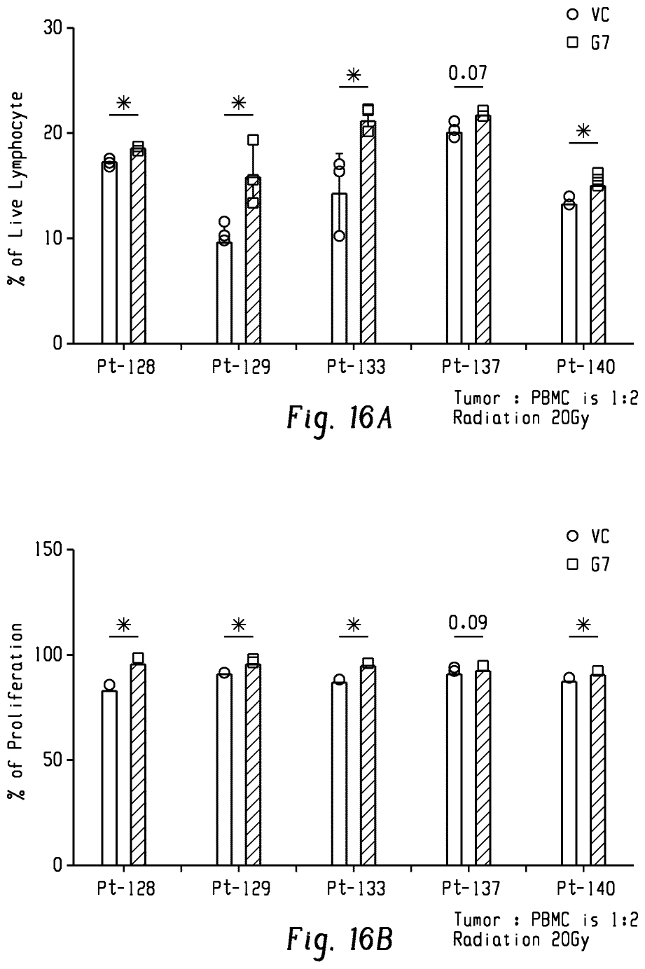

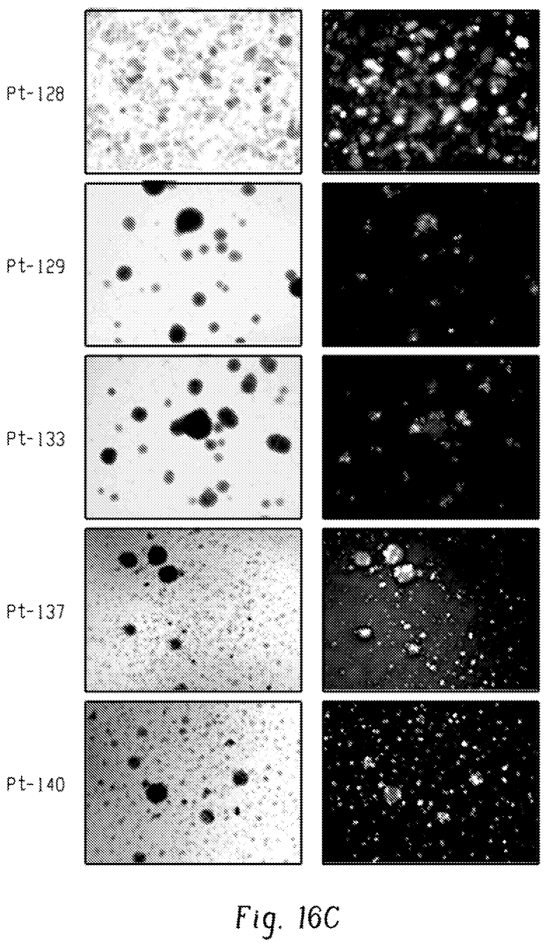

In the clinical setting, tumor vaccines can only be administered after a tumor is diagnosed and treated with the initial standard of care. GBM standard of care consists of surgical resection, followed by radiation+/−chemotherapy to manage the often-microscopic residual disease. To replicate the clinical treatment paradigm, a clinically relevant scenario was explored where a standard GL261 tumor, which was irradiated (50 gy) to simulate the clinical standard of care in GBM, was implanted and allowed to establish tumor (visualized by bioluminescence imaging (BLI)). After tumor establishment was confirmed with BLI, mice were then peripherally vaccinated with GIFT-7TVax or VC and monitored for OS (FIG. 11A). Even in the post tumor implantation vaccination model, the group vaccinated with GIFT-7TVax has significantly better OS compared to the VC (OS: GIFT-7TVax Vs VC 49 days Vs undefined) with survivors demonstrating complete tumor clearance via BLI (FIG. 11B). To make the tumor vaccination model translatable to human clinical trials, such as the previously trialed GM-CSF tumor vaccine GVAX, an irradiated GIFT-7TVax was generated. VC or GIFT-7 transfected tumor cells were radiated (50 Gy) prior to flank vaccination. To ensure radiation did not affect the fusokine production or viability of the GIFT-7TVax cells, a cell killing assay as well as ELISA post-radiation were performed. ELISA demonstrated continued IL-7 and GM-CSF secretion by the irradiated GIFT-7TVax cells compared to irradiated VC cells (GM-CSF: VC Vs GIFT-7:3.05 μg/ml to 354.5 μg/ml p<0.0001; IL-7: VC Vs GIFT-7:0.0 μg/ml to 1716 μg/ml p<0.0001) (FIG. 11C). The cell viability assay confirmed presence of viable cells in both VC and GIFT-7 group post radiation (No Rad vs Rad: VC 100% to 50.1% live p<0.0001; G7: 100% to 40.5% live p<0.0001) (FIG. 11C). The mice were peripherally vaccinated similar to our initial experiment with irradiated GIFT-7TVax and monitored for survival. Interestingly, irradiated GIFT-7TVax resulted in complete intracranial tumor clearance in 100% of the mice compared to 50% long-term survivors in the non-radiated GIFT-7TVax (MS no vax: 20 days, VC: 23 days, G7: 110.5 days, radG7: undefined) (FIG. 11D). TCR sequencing analysis was also conducted on mice who received irradiated GIFT-7TVax to determine the impact of the radiation on the T-cell repertoire. Productive frequency across the top 20 clones of each group showed a large reduction compared to both VC and GIFT-7Tvax, with similar productive frequency as the LTS mice from non-irradiated group (FIG. 11E). CDR3 transcript length remained similar between all the groups (FIG. 11F) as did productive TCRβ gene usage (FIG. 11G). Clone overlap between groups was also minimal as measured by Morisita Index as well as raw overlap (FIGS. 12A, B and C). Finally, raw nucleotide overlap between the 4 groups also indicated low similarity with only one sequence being represented in every group (FIG. 12D). Interestingly the irradiated GIFT-7Tvax did result in significantly more unique nucleotides than either GIFT-7TVax or LTS mice. (FIG. 12D) To fully establish the clinical translation potential of this treatment modality, GBM cells isolated from fresh tumor samples from patients after surgical resection (obtained from UW Hospital) were transfected and their ability to synthesize and secrete the GIFT-7 fusokine was validated (P95b: VC Vs G7: 0 μg/ml Vs 590 μg/μl p<0.0001; P96b: 7 pg/μl Vs 587 μg/μl p<0.0001) (FIG. 12E). These results demonstrate the relevance and efficacy of GIFT-7TVax vaccination in clinically applicable therapeutic scenarios, establishing the foundation for translation into a phase I clinical trials.

### Discussion

Increasingly, age related immune senescence is being recognized as an independent negative prognostic factor in many disease processes, however treatments tailored specifically to an aged population are rare. In the context of GBM, nearly all of the current clinical immunotherapies; DC-vaccination, immune-checkpoint blockade, CAR-T cell therapy, require a well-functioning immunological baseline to generate effective and durable adaptive anti-tumor immune response. It has been demonstrated repeatedly that there is dramatically reduced vaccine response and vaccine longevity in elderly due to alerted adaptive immune response. Furthermore, we and others have shown that by driving immune senescence GBM may itself accelerate age-related immune dysfunction. The culmination of these processes results in the most successful responders to GBM therapies, conventional or novel, being young individuals.

A fundamental observation made in this study was that a peripheral vaccination strategy works differently in young compared to aged hosts. Young mice given a peripheral vaccination without GIFT-7 were able to successfully clear intracranial tumor, however, aged mice demonstrated no peripheral vaccination effect without GIFT-7. Currently, the majority of GBM pre-clinical mouse work is done on 6-8 week old mice, which does not capture the effect of aging immune system seen in the GBM patient population. Because of this the age of the host should be taken into account when designing experiments, especially ones that rely on the baseline robustness of the immune system. Since all experiments were started in mice aged 52 weeks, many of the LTS mice experiments were done on mice over 80 weeks old and even in this population GIFT-7TVax generated lasting immunity. The data demonstrates an effective and durable tumor vaccination strategy, mediated by the fusokine GIFT-7, against a fatally incurable cancer uniquely in an aged model. GIFT-7TVax is efficacious in multiple GBM models in prolonging survival but is durable in the more immunogenic GL261 model. GL261 is a widely used and accepted syngeneic mouse model of glioma and is ideally suited for pre-clinical proof of concept studies of anti-tumor T-cell response due to its higher mutational burden, compared to human GBM. In this model, GIFT-7TVax regenerated the aged thymus resulting in increased T-cell recruitment and trafficking to the brain at the onset of the anti-tumor immune response. More importantly, it produced long-term survivors that displayed durable Th-17 long-term immunity in half of the mice treated. These LTS mice subsequently rejected contralateral tumor rechallenge 100% of the time. To make this vaccination strategy more clinically applicable, an irradiated GIFT-7TVax was created that continues to produce GIFT-7 with limited cell proliferation capabilities. This strategy of creating irradiated live tumor vaccine has been tested and proven safe in clinical trials. Remarkably, vaccination with irradiated GIFT-7TVax produces durable long-term immunity in 100% of the treated animals, likely due to increased immunogenicity of the irradiated tumor vaccine, which was evident from several unique TCR rearrangements seen in this group. In addition, the GIFT-7TVax strategy is also efficacious as a therapeutic vaccine when adapted to a more relevant clinical scenario in which mice with preestablished and treated tumors were vaccinated and monitored for survival.

Although the GIFT-7 fusokine was intended to augment the aged immune system, and its administration results in thymic regeneration and increased T-cell numbers, TCR repertoire analysis show that this T-cell augmentation is restricted in diversity and geared towards higher clonality likely due to tumor specific antigens from the cellular vaccination. This was well established by the sharp contrast in diversity and productivity of the GIFT-7TVax mice when compared to young non-tumor bearing mice. Indeed, in young mice we saw no significant benefit of GIFT-7TVax outside of the benefit of the VC peripheral tumor vaccination itself, further solidifying that GBM takes advantage of the limited functionality of the aged immune system. Since GIFT-7 combines IL-7, which primarily influences T-cell compartment, and GM-CSF, which influences dendritic cells, both the DC and T-cell compartments interrogated and it was found that GIFT-7 but not its individual component cytokines leads to the formation of hyperactive DCs. Hyperactive DCs are known to be more efficient at migration and antigen presentation and generate long lasting durable anti-tumor immunity. In the setting of systemic tumor hyperactive DCs are known to polarize anti-tumor immune response towards Th-1 response, however, in the intracranial tumor model Th-17 polarized long-lasting durable memory T-cells. This represents a possible division between the immunity generated against cancers that are accessible to the peripheral immune system versus cancers that are accessible only to the immune system patrolling the central nervous system (CNS). In fact, recent research has begun solidifying both the complexity and the uniqueness of the CNS immune system, so a difference in their responses to cancer or invasion in general is quite likely. In exploring this effect further, the TCR repertoire analysis of the productive V (D) J rearrangements in the LTS mice suggests a γδ mediated Th-17 specific effector phenotype induction could be driving this phenomenon. This population may be ultimately responsible for the long-term immunity conferred within the LTS group.

In summary, described here is the feasibility of targeting a therapy towards the aged immune system. The data highlights that accounting for age in the animal models of disease is critical. The GIFT-7TVax model was able to generate lasting anti-tumor immunity while still remaining effective when translated to more clinically relevant scenarios.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. An immunogenic composition comprising
a tumor cell expressing a fusokine comprising GM-CSF linked to IL-7 by a peptide linker.

2. The immunogenic composition of claim 1, wherein the fusokine comprises a sequence having 95% or greater sequence identity with SEQ ID NO: 1.

3. The immunogenic composition of claim 1, wherein the fusokine has the sequence of SEQ ID NO: 1.

4. The immunogenic composition of claim 1, wherein the tumor cell comprises a lentiviral or AAV vector expressing the fusokine.

5. The immunogenic composition of claim 1, wherein the tumor cell comprises an mRNA for expression of the fusokine.

6. The immunogenic composition of claim 1, wherein the tumor cell is an autologous tumor cell line, an allogenic tumor cell line, or an autologous cell from a tumor sample from the patient.

7. The immunogenic composition of claim 1, wherein the tumor cell is non-proliferating.

8. The immunogenic composition of claim 7, wherein the tumor cell has been irradiated or subjected to gene editing to prevent proliferation.

9. A pharmaceutical composition comprising the immunogenic composition of claim 1 and a pharmaceutically acceptable carrier.

10. A method of treating a patient in need of treatment for a glioma, comprising administering the pharmaceutical composition of claim 9.

11. The method of claim 10, wherein the glioma is a glioblastoma and the patient is human patient who is 64 years of age or older.

12. The method of claim 10, wherein administering is a peripheral injection.

13. The method of claim 12, wherein the injection is a subdermal or intramuscular injection.

14. The method of claim 10, wherein the patient is a human patient.

15. The method of claim 10, wherein the glioblastoma has previously been treated with surgical resection, followed by radiation and optionally chemotherapy.

16. The method of claim 10, wherein treating reduces intracranial tumor growth, provides an anti-tumor immune response (increase in CD3+ T-cells, CD4+ T-cells, CD8+ T-cells, effector memory T-cells, Il-2, Il-4, IL-1β, Caspase-1, NLRP3), or an provides an increase in overall survival.


SEQUENCE LISTING (2 sequences)

SEQ ID NO: 1 - protein, 325 aa, synthetic construct
  source 1..325: note = GIFT-7
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQESPVNMFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 2 - DNA, 978 bp, synthetic construct
  source 1..978: mol_type = other DNA; note = GIFT-7 coding sequence
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtcaccggtcaacatgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacactga